(19) United States
(12) Patent Application Publication
(10) Pub. No.: US 2011/0171135 A1
(43) Pub. Date: Jul. 14, 2011

(54) PEST-CONTROL COMPOSITIONS AND METHODS HAVING HIGH TARGET AND LOW NON-TARGET ACTIVITY

(76) Inventor: Essam Enan, Davis, CA (US)

(21) Appl. No.: 13/055,429

(22) PCT Filed: Jul. 22, 2009
(86) PCT No.: PCT/US09/51457

§ 371 Date: Mar. 30, 2011

Related U.S. Application Data
(60) Provisional application No. 61/082,601, filed on Jul. 22, 2008.

(51) Int. Cl. A61K 49/00

(52) U.S. Cl. 424/9.2

(57) ABSTRACT

Embodiments of the invention relate to compositions and methods for selective pest control wherein the compositions include active agents that in combination have a first activity against a selected target pest and a second activity against a selected non-target organism and wherein the first activity is greater than the second. Further embodiments of the invention relate to a method for developing selective pest control compositions and low-resistance pest control compositions.

### CROSS REFERENCE TO RELATED APPLICATIONS

This application claims priority to U.S. Provisional Patent Application No. 61/082,601, filed on Jul. 22, 2008, the entire contents of which are incorporated herein by reference.

### FIELD OF THE INVENTION

The present invention generally relates to pesticidal methods and compositions that minimally impact non-target species while providing highly selective control of target pest species, as well as reducing the likelihood of resistance development by the target species.

### BACKGROUND

The toxic effect of a pesticide upon both target- and non-target species can be evaluated using LD₅₀ values. An LD₅₀ represents the dose required to kill 50 percent of a population of test animals (e.g., rats, fish, mice, cockroaches). The lower the LD₅₀, the more potent the pesticide, and less is required to kill a target pest; i.e., a pesticide with an LD₅₀ value of 10 mg/kg is 10 times more toxic than a pesticide with an LD₅₀ of 100 mg/kg. The non-target toxicity of an pesticide is important in helping to determine potential hazards associated with its use, and preferably the mode of action for a particular pesticide is such that its LD₅₀ for non-target species is much higher than that for non-target species, in both a broad (vertebrate/invertebrate) and narrow (between different species of pest) sense.

Many chemical pesticides, to a greater or lesser extent, exert a selective evolutionary pressure upon the pests they are intended to control. Thus, over a period of time, resistant strains of pests can emerge, as application of the pesticide will kill non-resistant pests and spare the resistant ones. Resistance development depends on a number of factors, including the frequency and nature of resistance alleles, pest management strategies, and the relative fitness of the resistant strains relative to the wild type. This can be especially troublesome with regard to insects, as their large populations and short generation times can result in rapid development of resistance to particular pesticides. Currently, approximately 500 species of insect pest are resistant to one or more common insecticides, and this number is increasing as shown in FIG. 1.

### SUMMARY OF THE INVENTION

Embodiments of the invention provide a selective pest-control composition having at least two active agents, wherein the active agents in combination have a first activity against a selected target pest, and wherein the active agents in combination have a second activity against a selected non-target organism, and wherein the first activity is greater than the second activity.

In further aspects, the first activity is at least 2, 10, 20, 50, or 100 times greater than the second activity.

In a further aspect, the first activity results from interaction of the composition with at least one biological target that is substantially absent in the non-target organism.

In a further aspect, the non-target organism is selected from the group consisting of vertebrate animals and plants.

In a further aspect, the non-target organism is a vertebrate animal, the ratio of the first activity to the second activity is expressed as a ratio of LD50(target) to LD50(vertebrate animal), and the ratio is less than 0.1.

In a further aspect, the first activity results from interaction of the composition with at least one biological target that is present in the non-target organism.

In a further aspect, the biological target is a G protein-coupled receptor.

In a further aspect, the G-protein coupled receptor is selected from the group consisting of a tyramine receptor, an octopamine receptor, olfactory receptor Or83b, and olfactory receptor 43a.

In a further aspect, the active agents have a synergistic activity in the target pest.

In a further aspect, the active agents have a non-synergistic activity or antagonistic activity in the non-target organism.

Further embodiments of the invention provide a method of selective pest control comprising contacting a target pest with a composition comprising at least two active agents, wherein the active agents in combination have a first activity against the target pest, and a second activity against a non-target organism, and wherein the first activity is 10 times greater than the second activity.

Further embodiments of the invention provide a method of developing a selective pest control composition, comprising the steps of: selecting a target pest for selective control by the composition, and a non-target organism to be substantially unaffected by the composition; identifying at least two active agents for the composition, wherein the active agents in combination have a complementary effect within the target pest; and confirming that the identified active agents do not have a complementary effect within the non-target organism, to establish that the composition is selective of the target pest over the non-target organism.

In a further aspect, the complementary effect comprises a synergistic effect of the active agents together as compared with an effect of each active agent separately.

In a further aspect, the confirming step comprises confirming that the agents in combination have an effect in the non-target organism selected from the group consisting of: an antagonistic effect, a non-additive effect, and an additive effect.

Further embodiments of the invention provide a method of developing a low-resistance pest control formulation against a target pest, comprising the steps of: selecting a target pest, the pest having at least a first and a second molecular target, where the molecular targets are under genetic control; selecting at least two active agents, wherein a first active agent interacts with the first molecular target under genetic control and a second active agent interacts with the second molecular target under genetic control; and combining the two active agents in a formulation, wherein the agents in the formulation act upon the target pest in a complementary manner, and wherein resistance to the formulation in an individual target pest requires two separate genetic lesions divergent from a non-resistant population of the pest.

In a further aspect, the first and second molecular targets comprise two separate molecules encoded or controlled by separate genetic elements.

In a further aspect, the first molecular target is controlled by a first genetic element encoding a protein, the second molecular target is controlled by a second genetic element encoding a protein, and the two separate genetic lesions comprise a lesion in the first genetic element and a lesion in the second genetic element.

In a further aspect, the first molecular target is a cellular entity, the second molecular target is a cellular entity, and the two separate genetic lesions comprise a lesion in the first genetic element and a lesion in the second genetic element.

In a further aspect, the first and second molecular targets comprise two separate portions of a single molecule encoded or controlled by a single genetic element, and wherein the two separate portions are at a distance from each other such that conversion of each of the two separate portions to a resistant form would require two separate genetic lesions within the single genetic element.

In a further aspect, the complementary manner comprises a synergistic effect as compared with each agent acting separately.

### BRIEF DESCRIPTION OF THE DRAWINGS

FIG. 1 shows the development of insect resistance to various insecticide classes over time.

FIG. 2 shows flea mortality following treatment with test compositions.

FIG. 3 shows tick mortality following treatment with test compositions.

FIG. 4 shows the percent mortality of various chemicals on both wild type and mutant Drosophila.

FIG. 5 shows the anti-parasitic effect of test compositions.

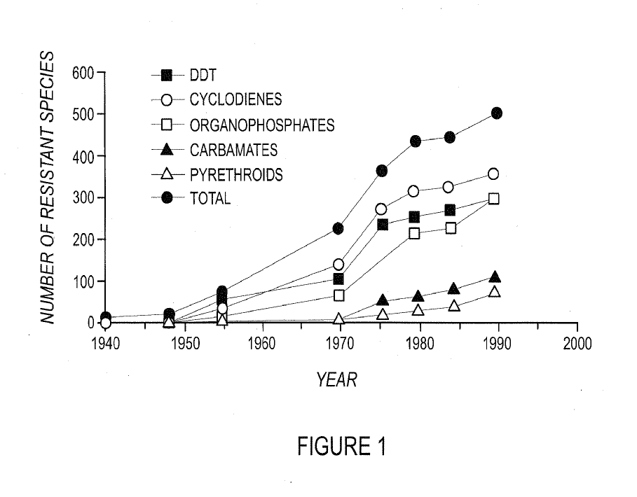

### DETAILED DESCRIPTION OF THE INVENTION

Many commercialized products having sufficient pesticidal activity to be useful also have toxic or deleterious effects on mammals, fish, fowl, plants, or other non-target species. For example, common insecticides such as organophosphorus compounds and carbamates inhibit the activity of acetylcholinesterase in all classes of animals. Chlordimeform and related formamidines are known to act on insect octopamine receptors, but have been removed from the market because of cardiotoxic potential in vertebrates and carcinogenicity in animals and a varied effect on different insects. Thus, pesticide selectivity, or the ability to target certain species while minimally affecting others, is of critical importance. Compositions according to embodiments of the invention can selectively control insects of a target species while minimally affecting vertebrates or plants. Likewise, embodiments can selectively control insects of a target species while minimally affecting non-target invertebrates. Furthermore, embodiments can selectively control insects of a target species while minimally affecting non-target insect species.

In some embodiments, exposure to the blend contained in the pest control agent disrupts cellular calcium levels within the target organism, and/or exposure to the pest control agent disrupts cyclic AMP levels within cells of the target organism. In some embodiments, exposure to the blend can result in binding of a receptor of the olfactory cascade of the target organism. In some embodiments, one or more components of the blend can act as an agonist or antagonist on the receptor of the target organism. Some blends include at least three active ingredients, or at least four active ingredients. In other embodiments, exposure to the blend can disrupt cellular events without targeting the receptor.

As used herein, “pests” mean any organism the control of which is desired. Pests can include, for example, bacteria, fungi, insects, soil nematodes, animal and human parasites such as cestodes, trematodes, nematodes, and the like, protozoa, plants, and the like.

As used herein, “pesticidal” refers to the killing of pests, and means, for example, antibacterial, antifungal, antiparasitic, herbicidal, insecticidal, and the like, depending on the type of pest in question.

As used herein, a “biological target” is any biological structure or process that can be acted upon by an active ingredient, and includes, for example, an organ, tissue, a cell, a receptor, a cellular or extracellular molecule, a process, a cascade/pathway, or the like.

As used herein, a “receptor” is an entity on the cell membrane or within the cell, cytoplasm, or cell nucleus, that can bind to a specific molecule (a ligand), such as, for example, a neurotransmitter, hormone, or the like, and initiate the cellular response to the ligand.

As used herein, “tyramine receptor” can mean any tyramine receptor of non-vertebrate origin.

As used herein, the term “insect control” means the presence of a repellant effect, a pesticidal effect, or both that is attributable to the presence of an active ingredient.

As used herein, a “repellant effect” is an effect, wherein more insects are repelled away from a host or area that has been treated with the composition than a control host or area that has not been treated with the composition. In some embodiments, repellant effect is an effect wherein at least about 15% of insects are repelled away from a host or area that has been treated with the composition. In some embodiments, repellant effect is an effect wherein at least about 90% of insects are repelled away from a host or area that has been treated with the composition.

As used herein, a “pesticidal effect” is an effect, wherein treatment with a composition causes at least about 1% of the insects to die. In this regard, an LC1 to LC100 (lethal concentration) or an LD1 to LD100 (lethal dose) of a composition will cause a pesticidal effect.

As used herein, LD₅₀ represents the dose required to kill 50 percent of a population of test animals. The LD₅₀, of a target pest versus a non-target can be expressed as a ratio.

For example a ratio of the LD₅₀ for a target pest to the LD₅₀ of a non-target of 0.1 would indicate that the effect on the target pest is 10 times the effect on the non-target species.

As used herein, “host” can mean a plant, human or other animal.

As used herein, “compositions” includes single- and multi-component mixtures. “Compositions” can include the individual components of a mixture or any subset of individual components, such as, for example, a fraction of a mixture as separated by any of a variety of methods known in the art.

As used herein, “antagonistic activity” is shown when the pesticidal activity of a blend of components is less than the individual activity of the blend's most-active component.

As used herein, “sub-additive activity” means that the pesticidal activity of a blend of components is less than the sum of the components' individual activities, but more than the individual activity of the blend's most-active component.

In embodiments of the present invention, “synergy” is a specific feature of a combination of ingredients, and is shown by a measurable effect of a combination of active ingredients that is above any background level of enhancement that would be due solely to, e.g., additive effects of that combination of ingredients. Effects that may be used to show synergy include but are not limited to: repellant effect of the composition; pesticidal effect of the composition; perturbation of a cell message or cell signal such as, e.g., calcium, cyclic-AMP, and the like; and diminution of activity or downstream effects of a molecular target.

In various embodiments, a substantial enhancement can be expressed as a coefficient of synergy, wherein the coefficient is a ratio of the measured effect of the complete blend, divided by the effect of a comparison composition, typically a single ingredient or a subset of ingredients found in the complete blend. In some embodiments, the synergy coefficient can be adjusted for differences in concentration of the complete blend and the comparison composition.

In some embodiments of the invention, a coefficient of synergy of 1.1, 1.2, 1.3, 1.4, or 1.5 can be substantial and commercially desirable. In other embodiments, the coefficient of synergy can be from about 1.6 to about 5, including but not limited to 1.8, 2.0, 2.5, 3M, 3.5, 4.0, and 4.5. In other embodiments, the coefficient of synergy can be from about 5 to 50, including but not limited to 10, 15, 20, 25, 30, 35, 40, and 45. In other embodiments, the coefficient of synergy can be from about 50 to about 500, or more, including but not limited to 50, 75, 100, 125, 150, 200, 250, 300, 350, 400, and 450. Any coefficient of synergy above 500 is also contemplated within embodiments of the present invention.

Given that a broad range of synergies can be found in various embodiments of the invention, it is expressly noted that a coefficient of synergy can be described as being “greater than” a given number and therefore not necessarily limited to being within the bounds of a range having a lower and an upper numerical limit. Likewise, in some embodiments of the invention, certain low synergy coefficients, or lower ends of ranges, are expressly excluded. Accordingly, in some embodiments, synergy can be expressed as being “greater than” a given number that constitutes a lower limit of synergy for such an embodiment. For example, in some embodiments, the synergy coefficient is equal to or greater than 25; in such an embodiment, all synergy coefficients below 25, even though substantial, are expressly excluded.

Compositions containing combinations of certain chemicals and compounds can be tested for synergistic effect on insect control activity by comparing the effect of a particular combination of at least one chemical, and at least one compound or at least one blend of compounds, to the effect of the individual chemical(s) and compound(s).

In certain embodiments, the invention provides methods of making a synergistic pest control formulation having desirable environmental properties. The methods can include the steps of: selecting an ingredient from a group of candidate ingredients known or believed to be generally safe for use in contact with vertebrates; screening the ingredient for binding to a G protein-coupled receptor of an invertebrate, that is, the binding results in measurable disruption of cellular calcium or cyclic AMP; combining the screened ingredient with at least one other screened ingredient. Desirably, the ingredients, in combination, are synergistic in an effect against a target organism. The receptor can be a receptor of the insect olfactory cascade, including, for example, a tyramine receptor, an octopamine receptor, olfactory receptor Or83b, olfactory receptor 43a, and the like. Other receptors that may be employed include serotonin receptor, Or22a, Or22b, Gr5a, Gr21a, Gr61a, β-arrestin receptor, GRK2 receptor, tyramine β-hydroxylase receptor, and the like.

Exemplary methods that can be used to determine the synergistic effect of a particular composition are set forth in the following applications, each of which is incorporated in its entirety herein by reference: U.S. Pat. No. 7,541,155, entitled COMPOSITIONS AND METHODS FOR CONTROLLING INSECTS; U.S. application Ser. No. 11/086,615, entitled COMPOSITIONS AND METHODS FOR CONTROLLING INSECTS RELATED TO THE OCTOPAMINE RECEPTOR; U.S. application Ser. No. 11/365,426, entitled COMPOSITIONS AND METHODS FOR CONTROLLING INSECTS INVOLVING THE TYRAMINE RECEPTOR; U.S. application Ser. No. 11/870,385, entitled COMPOSITIONS AND METHODS FOR CONTROLLING INSECTS; and U.S. application Ser. No. 12/009,220, entitled PEST CONTROL COMPOSITIONS AND METHODS. Embodiments of the invention can provide a method of invertebrate control, including providing a composition comprising at least two active ingredients. Desirably, the at least two active ingredients are ligands of a G-protein coupled receptor in a target invertebrate; and when contacted with the invertebrate, the composition results in synergistic invertebrate control.

This screening will be briefly described using the example of the tyramine receptor. However, the methods described would be adaptable by one of skill in the art to any of the other receptors described in the present application. For example, in some embodiments, other receptors, such as G-protein coupled receptors (GPCRs), whether having native affinity for tyramine or other ligands, can be employed in methods of screening for compositions useful for treating a parasitic infection. Examples of receptors that can be used include, but are not limited to: Anopheles gambiae (GAN: EAA07468), Heliothis virescens (GAN: Q25188), Mamestra brassicae (GAN: AAK 14402), Tribolium castaneum (GAN: XP_{—}970290), Aedes aegypti (GAN: EAT41524), Boophilus microplus (GAN: CAA09335); Schistosoma mansoni (GAN: AAF73286); and Schistosoma mansoni (GAN: AAW21822). In other embodiments, receptors of the nuclear hormone receptor superfamily can be employed in methods of screening for compositions useful for treating a parasitic infection. Examples of receptors that can be used include, but are not limited to receptors from parasites or invertebrates that are analogous to the DAF family of nuclear receptors such as DAF-2 and DAF-12. In other embodiments, nuclear receptor proteins from Drosophila or other invertebrate can be employed, such as: nuclear receptors of subfamily 1 such as E78, E75, DHR3, EcR, and DHR96; nuclear receptors of subfamily 2 such as USP, DHR78, HNF4, SVP, TLL, DSF, DHR51, or DHR83; nuclear receptors of subfamily 3 such as ERR, nuclear receptors of subfamily 4 such as DHR38; nuclear receptors of subfamily 5 such as FTZ-F1 or DHR39; or nuclear receptors of subfamily 6 such as DHR4. In other embodiments, invertebrate or parasite nuclear receptor proteins analogous to certain human nuclear receptors can be employed, such as: nuclear receptors of subfamily 1 such as PPAR, RAR, TR, REV-ERB, ROR, FXR, LXR, VDR, SXR, or CAR; nuclear receptors of subfamily 2 such as RXR, TR2/TR4, HNF4, COUP-TF, TLX, or PNR; nuclear receptors of subfamily 3 such as ERR, ER, or MR/PR/AR/GR; nuclear receptors of subfamily 4 such as NURRI/NGFIB; nuclear receptors of subfamily 5 such as LRH/SFl; or nuclear receptors of subfamily 6 such as GCNF. In other embodiments, invertebrate or parasite nuclear receptor proteins having as their native ligand naturally occurring hormones such as la, 25(OH)2-vitamin D3, 17p-oestradiol, testosterone, progesterone, cortisol, aldosterone, all-trans retinoic acid, 3,5,3′-L-triiodothyronine, cc-ecdysone, or brassinolide, among others, can be employed.

The cells used for the method can be any cell capable of being transfected with and express a Tyramine Receptor (TyrR). Examples of cells include, but are not limited to: insect cells, such as Drosophila Schneider cells, Drosophila Schneider 2 cells (S2 cells), and Spodoptera frugiperda cells (e.g., Sf9 or Sf21); or mammalian cells, such as Human Embryonic Kidney cells (HEK-293 cells), African green monkey kidney fibroblast cells (COS-7 cells), HeLa Cells, and Human Keratinocyte cells (HaCaT cells).

The tyramine receptor (TyrR) can be a full-length TyrR, a functional fragment of a TyrR, or a functional variant of a TyrR. A functional fragment of a TyrR is a TyrR in which amino acid residues are deleted as compared to the reference polypeptide, i.e., full-length TyrR, but where the remaining amino acid sequence retains the binding affinity of the reference polypeptide for tyramine. A functional variant of a TyrR is a TyrR with amino acid insertions, amino acid deletions, or conservative amino acid substitutions, which retains the binding affinity of the reference polypeptide for tyramine. Examples of TyrR5 include, but are not limited to Drosophila melanogaster TyrR (GENBANK® accession number (GAN) CAA38565), Locusta migratoria TyrR (GAN: □25321), TyrR5 of other invertebrates, and TyrR5 of nematodes, including Ascaris.

Tyramine receptor may be amplified, for example, from a Drosophila melanogaster head cDNA phage library. Phage DNA may be purified from this library by known methods using a liquid culture lysate. The open reading frame of the Drosophila tyramine receptor (TyrR) may be amplified via PCR, and the PCR product may be digested, and the TyrR open reading frame may be inserted into an expression vector using known methods, and transfected Schneider cell clones may be prepared.

Tyramine receptor binding/uptake may be performed to determine which of the transfected clones have the highest levels of functionally active tyramine receptor protein, and the selected clone may be propagated and stored in liquid nitrogen. Aliquots of the selected clone are grown for whole cell binding and for plasma membrane preparation for kinetic and screening studies.

For binding studies, cells transfected with the tyramine receptor (about 1×10⁶ cells/ml) are cultured in each well of a multi-well plate, and binding is assessed using ³H-tyramine and unlabeled tyramine. Maximum specific binding occurs at about 5 nM ³H-tyramine, and untransfected cells do not respond to tyramine at concentration as high as about 100 μM. Saturation binding experiments may be performed to determine Bₘₐₓ and K_{d} values for TyrR. Further study of affinities of other neurotransmitter ligands (such as octopamine, dopamine, and serotonin) for TyrR shows that Schneider cells expressing tyramine receptor are effective as a model for studies and screening for compositions that interact with the tyramine receptor.

Evaluation of the secondary messenger cAMP may be based on competition binding between endogenous cAMP and ³H-cAMP to a cAMP binding protein by known methods. Intracellular calcium ion concentrations ([Ca²⁺]i) may be measured by using the acetoxymethyl (AM) ester of the fluorescent indicator fura-2 by known methods. A cell suspension may be incubated with Fura 2/AM, and the cells then pelleted and resuspended. [Ca²⁺]i changes may be analyzed in spectrofluorometer in the presence and absence of test chemicals.

Treatment with certain plant essential oils, including those expressly set forth in the application, result in changes in intracellular cAMP or Ca²⁺ levels in cells expressing tyramine receptor, and this can serve as a screening method for evaluating compositions that have potential as selective pest control compositions or pest control compositions unlikely to lead to the development of resistance.

The selective pest control compositions of the present invention may rely for their effect on the substantial absence of the receptors used in the screening process, or closely related receptors, in non-target organisms. For example, trace amine receptors such as TyrR or octopamine receptor are substantially absent from vertebrates. In this context, “substantially absent” means that the receptors are present, if at all, in such a low concentration that no discernible effects are observed at concentrations at which intracellular secondary messenger perturbation and observable effects are observed in invertebrates that express the receptor. Alternatively, the selective pest control compositions of the present invention may exhibit differential effects on different invertebrates despite the presence in both invertebrates of receptors of the type employed in the screening method described above. These differential effects may result from differences in the amino acid sequences of the related receptors in the different species or from differential expression patterns of the receptors in the issues of the invertebrates.

In some methods of embodiments of the invention, the control comprises repulsion of substantially all of the target pests or invertebrates, and in some the control comprises knockdown of substantially all of the target pests or invertebrates, and in others the control comprises killing of substantially all of the target pests or invertebrates.

The details of one or more embodiments of the invention are provided. Modifications to embodiments described in this document, and other embodiments, will be evident to those of ordinary skill in the art after a study of the information provided in this document. The information provided in this document, and particularly the specific details of the described exemplary embodiments, is provided primarily for clarity of understanding and no unnecessary limitations are to be understood therefrom.

Embodiments of the invention can include compositions for controlling insects, and methods for making and using these compositions. Certain embodiments of the invention can include one or more plant essential oils. Additionally, in preferred embodiments, these formulations can be made up of generally regarded as safe (GRAS) compounds.

Compositions according to embodiments of the invention can be applied as liquids, suspension, emulsions, or solids by conventional application techniques for each.

In some embodiments of the present invention, the insect-control composition can be, for example, a composition including one or more plant essential oils, such as, for example, the compositions listed in Table 1. In the table “LFO” designates lilac flower oil and “BSO” designates black seed oil.

TABLE 1
BLENDS

| | | CAS Registry | | |
|---|---|---|---|---|
| | Compounds | Number | low % | high % |
| Blend 1 | LFO | | 4 | 30 |
| | D-Limonene | 5989-27-5 | 8 | 99 |
| | Thyme Oil White | 8007-46-3 | 0.1 | 20 |
| | Blend 65 | | 8 | 99 |
| Blend 2 | D-Limonene | 5989-27-5 | 9 | 99 |
| | Thyme Oil White | 8007-46-3 | 0.1 | 20 |
| | Linalool Coeur | 78-70-6 | 0.1 | 4 |
| | Tetrahydrolinalool | 78-69-3 | 0.1 | 5 |
| | Vanillin | 121-33-5 | 0.06 | 0.3 |
| | Isopropyl myristate | 110-27-0 | 0.1 | 5 |
| | Piperonal (aldehyde) [Heliotropine] | 120-57-0 | 0.1 | 5 |
| | Blend 66 | | 8 | 99 |
| | Geraniol Fine FCC | 106-24-1 | 0.1 | 4 |
| | Triethyl Citrate | 77-93-0 | 0.1 | 5 |
| Blend 3 | D-Limonene | 5989-27-5 | 45 | 99 |
| | Thyme Oil White | 8007-46-3 | 0.1 | 10 |
| | Blend 66 | | 5 | 30 |
| | Blend 63 | | 0.1 | 10 |
| Blend 4 | LFO | | 30 | 99 |
| | BSO | 977017-84-7 | 15 | 99 |
| Blend 5 | BSO | 977017-84-7 | 15 | 99 |
| | Linalool Coeur | 78-70-6 | 6 | 40 |
| | Tetrahydrolinalool | 78-69-3 | 8 | 45 |
| | Vanillin | 121-33-5 | 0.1 | 5 |
| | Isopropyl myristate | 110-27-0 | 10 | 55 |
| | Piperonal (aldehyde) [Heliotropine] | 120-57-0 | 0.1 | 20 |
| | Geraniol Fine FCC | 106-24-1 | 0.1 | 25 |
| Blend 6 | D-Limonene | 5989-27-5 | 0.1 | 25 |
| | BSO | 977017-84-7 | 15 | 85 |
| | Linalool Coeur | 78-70-6 | 0.1 | 25 |
| | Tetrahydrolinalool | 78-69-3 | 0.1 | 25 |
| | Vanillin | 121-33-5 | 0.1 | 3 |
| | Isopropyl myristate | 110-27-0 | 0.1 | 30 |
| | Piperonal (aldehyde) [Heliotropine] | 120-57-0 | 0.1 | 10 |
| | Geraniol Fine FCC | 106-24-1 | 0.1 | 15 |
| | Methyl Salicylate 98% Nat | 119-36-8 | 8 | 70 |
| Blend 7 | Thyme Oil White | 8007-46-3 | 15 | 90 |
| | Wintergreen Oil | 68917-75-9 | 15 | 99 |
| | Vanillin | 121-33-5 | 0.1 | 4 |
| | Isopropyl myristate | 110-27-0 | 20 | 99 |
| Blend 8 | D-Limonene | 5989-27-5 | 20 | 99 |
| | Thyme Oil White | 8007-46-3 | 0.1 | 25 |
| | Wintergreen Oil | 68917-75-9 | 25 | 99 |
| Blend 9 | LFO | | 6 | 40 |
| | D-Limonene | 5989-27-5 | 25 | 99 |
| | Thyme Oil White | 8007-46-3 | 5 | 30 |
| | Linalool Coeur | 78-70-6 | 0.1 | 3 |
| | Citral | 5392-40-5 | 0.1 | 20 |
| | gamma-terpinene | 99-85-4 | 0.1 | 20 |
| | Alpha-Pinene, 98% | 80-56-8 | 0.1 | 5 |
| | alpha-Terpineol | 98-55-5 | 0.1 | 15 |
| | Terpinolene | 586-62-9 | 0.1 | 15 |
| | Para-Cymene | 99-87-6 | 0.1 | 5 |
| | Linalyl Acetate | 115-95-7 | 0.1 | 6 |
| | Beta Pinene | 127-91-3 | 0.1 | 6 |
| | Camphor Dextro | 464-49-3 | 0.05 | 0.3 |
| | Terpinene 4 OL | 562-74-3 | 0.05 | 0.3 |
| | Alpha Terpinene | 99-86-5 | 0.1 | 6 |
| | Borneol L | 507-70-0 | 0.1 | 3 |
| | Camphene | 79-92-5 | 0.1 | 2 |
| | Decanal | 112-31-2 | 0.06 | 0.3 |
| | Dodecanal | 112-54-9 | 0.06 | 0.3 |
| | Fenchol Alpha | 512-13-0 | 0.005 | 0.1 |
| | Geranyl Acetate | 105-87-3 | 0.06 | 0.3 |
| | Isoborneol | 124-76-5 | 0.08 | 1 |
| | 2-Methyl 1,3-cyclohexadiene | 30640-46-1, 1888- | 0.08 | 1 |
| | | 90-0 | | |
| | Myrcene | 123-35-3 | 0.1 | 3 |
| | Nonanal | 124-19-6 | 0.005 | 0.08 |
| | Octanal | 124-13-0 | 0.005 | 0.2 |
| | Tocopherol Gamma (TENOX ®) | 54-28-4 | 0.005 | 0.08 |
| Blend 10 | D-Limonene | 5989-27-5 | 0.1 | 25 |
| | Thyme Oil White | 8007-46-3 | 0.1 | 25 |
| | Blend 65 | | 40 | 99 |
| | Linalool Coeur | 78-70-6 | 0.1 | 6 |
| | Tetrahydrolinalool | 78-69-3 | 0.1 | 8 |
| | Vanillin | 121-33-5 | 0.08 | 0.6 |
| | Isopropyl myristate | 110-27-0 | 0.1 | 8 |
| | Piperonal (aldehyde) [Heliotropine] | 120-57-0 | 0.1 | 8 |
| | Geraniol Fine FCC | 106-24-1 | 0.1 | 4 |
| | Triethyl Citrate | 77-93-0 | 0.1 | 8 |
| Blend 11 | Thyme Oil White | 8007-46-3 | 3 | 65 |
| | Wintergreen Oil | 68917-75-9 | 15 | 99 |
| | Isopropyl myristate | 110-27-0 | 20 | 99 |
| Blend 12 | D-Limonene | 5989-27-5 | 5 | 30 |
| | Linalool Coeur | 78-70-6 | 8 | 40 |
| | Tetrahydrolinalool | 78-69-3 | 15 | 99 |
| | Vanillin | 121-33-5 | 0.1 | 8 |
| | Isopropyl myristate | 110-27-0 | 15 | 85 |
| | Piperonal (aldehyde) [Heliotropine] | 120-57-0 | 5 | 30 |
| | Geraniol Fine FCC | 106-24-1 | 5 | 30 |
| Blend 13 | D-Limonene | 5989-27-5 | 5 | 30 |
| | Geraniol Fine FCC | 106-24-1 | 5 | 30 |
| | Blend 62 | | 50 | 99 |
| Blend 14 | D-Limonene | 5989-27-5 | 5 | 30 |
| | Blend 72 | | 55 | 99 |
| Blend 15 | D-Limonene | 5989-27-5 | 5 | 30 |
| | Linalool Coeur | 78-70-6 | 10 | 55 |
| | Tetrahydrolinalool | 78-69-3 | 10 | 65 |
| | Vanillin | 121-33-5 | 0.1 | 4 |
| | Isopropyl myristate | 110-27-0 | 10 | 60 |
| | Piperonal (aldehyde) [Heliotropine] | 120-57-0 | 10 | 65 |
| | Piperonyl Alcohol | 495-76-1 | 0.1 | 25 |
| Blend 16 | D-Limonene | 5989-27-5 | 5 | 30 |
| | BSO | 977017-84-7 | 15 | 80 |
| | Linalool Coeur | 78-70-6 | 5 | 30 |
| | Tetrahydrolinalool | 78-69-3 | 6 | 35 |
| | Vanillin | 121-33-5 | 0.1 | 4 |
| | Mineral Oil White (USP) | 8042-47-5 | 8 | 45 |
| | Isopropyl myristate | 110-27-0 | 8 | 45 |
| | Piperonal (aldehyde) [Heliotropine] | 120-57-0 | 0.1 | 15 |
| | Geraniol Fine FCC | 106-24-1 | 0.1 | 20 |
| Blend 17 | D-Limonene | 5989-27-5 | 10 | 99 |
| | Linalool Coeur | 78-70-6 | 0.1 | 10 |
| | Tetrahydrolinalool | 78-69-3 | 0.1 | 10 |
| | Vanillin | 121-33-5 | 0.08 | 0.6 |
| | Isopropyl myristate | 110-27-0 | 0.1 | 10 |
| | Piperonal (aldehyde) [Heliotropine] | 120-57-0 | 0.1 | 10 |
| | Piperonyl Alcohol | 495-76-1 | 0.1 | 5 |
| | Blend 66 | | 10 | 99 |
| Blend 18 | Linalool Coeur | 78-70-6 | 0.1 | 15 |
| | Tetrahydrolinalool | 78-69-3 | 0.1 | 20 |
| | Vanillin | 121-33-5 | 0.1 | 2 |
| | Isopropyl myristate | 110-27-0 | 0.1 | 20 |
| | Piperonal (aldehyde) [Heliotropine] | 120-57-0 | 0.1 | 20 |
| | Piperonyl Alcohol | 495-76-1 | 0.1 | 10 |
| | Blend 66 | | 40 | 99 |
| Blend 19 | LFO | | 20 | 99 |
| | D-Limonene | 5989-27-5 | 15 | 85 |
| | Thyme Oil White | 8007-46-3 | 15 | 90 |
| Blend 20 | D-Limonene | 5989-27-5 | 15 | 85 |
| | Thyme Oil White | 8007-46-3 | 15 | 95 |
| | Blend 63 | | 20 | 99 |
| Blend 21 | D-Limonene | 5989-27-5 | 15 | 85 |
| | Thyme Oil White | 8007-46-3 | 15 | 90 |
| | Linalool Coeur | 78-70-6 | 0.1 | 15 |
| | Tetrahydrolinalool | 78-69-3 | 0.1 | 25 |
| | Vanillin | 121-33-5 | 0.1 | 2 |
| | Isopropyl myristate | 110-27-0 | 0.1 | 25 |
| | Piperonal (aldehyde) [Heliotropine] | 120-57-0 | 0.1 | 25 |
| | Geraniol Fine FCC | 106-24-1 | 0.1 | 10 |
| | Triethyl Citrate | 77-93-0 | 0.1 | 25 |
| Blend 22 | Phenyl Ethyl Propionate | | 20 | 99 |
| | Methyl Salicylate | | 20 | 99 |
| | Blend 43 | | 15 | 85 |
| Blend 23 | D-Limonene | 5989-27-5 | 0.1 | 10 |
| | Thyme Oil White | 8007-46-3 | 0.1 | 15 |
| | Benzyl Alcohol | 100-51-6 | 8 | 50 |
| | Isopar M | 64742-47-8 | 10 | 65 |
| | Water | 7732-18-5 | 25 | 99 |
| | Blend 63 | | 0.1 | 15 |
| | Stock 10% SLS Solution | | 0.1 | 10 |
| Blend 24 | D-Limonene | 5989-27-5 | 0.1 | 10 |
| | Thyme Oil White | 8007-46-3 | 0.1 | 15 |
| | Linalool Coeur | 78-70-6 | 0.1 | 3 |
| | Tetrahydrolinalool | 78-69-3 | 0.1 | 4 |
| | Vanillin | 121-33-5 | 0.05 | 0.3 |
| | Isopropyl myristate | 110-27-0 | 0.1 | 4 |
| | Piperonal (aldehyde) [Heliotropine] | 120-57-0 | 0.1 | 4 |
| | Geraniol Fine FCC | 106-24-1 | 0.1 | 2 |
| | Triethyl Citrate | 77-93-0 | 0.1 | 4 |
| | Benzyl Alcohol | 100-51-6 | 8 | 50 |
| | Isopar M | 64742-47-8 | 10 | 65 |
| | Water | 7732-18-5 | 25 | 99 |
| | Stock 10% SLS Solution | | 0.1 | 10 |
| Blend 25 | D-Limonene | 5989-27-5 | 6 | 40 |
| | Thyme Oil White | 8007-46-3 | 8 | 45 |
| | Benzyl Alcohol | 100-51-6 | 30 | 99 |
| | Blend 63 | | 10 | 55 |
| Blend 26 | LFO | | 0.1 | 25 |
| | D-Limonene | 5989-27-5 | 8 | 99 |
| | Thyme Oil White | 8007-46-3 | 0.1 | 20 |
| | Blend 66 | | 8 | 99 |
| Blend 27 | Linalool Coeur | 78-70-6 | 0.1 | 20 |
| | Soy Bean Oil | 8016-70-4 | 10 | 70 |
| | Thymol (crystal) | 89-83-8 | 20 | 99 |
| | Alpha-Pinene, 98% | 80-56-8 | 0.1 | 10 |
| | Para-Cymene | 99-87-6 | 15 | 85 |
| Blend 28 | Linalool Coeur | 78-70-6 | 0.1 | 25 |
| | Thymol (crystal) | 89-83-8 | 25 | 99 |
| | Alpha-Pinene, 98% | 80-56-8 | 0.1 | 15 |
| | Para-Cymene | 99-87-6 | 20 | 99 |
| Blend 29 | D-Limonene | 5989-27-5 | 0.1 | 25 |
| | Thyme Oil White | 8007-46-3 | 0.1 | 30 |
| | Blend 65 | | 35 | 99 |
| | Linalool Coeur | 78-70-6 | 0.1 | 8 |
| | Tetrahydrolinalool | 78-69-3 | 0.1 | 10 |
| | Vanillin | 121-33-5 | 0.08 | 1 |
| | Isopropyl myristate | 110-27-0 | 0.1 | 10 |
| | Piperonal (aldehyde) [Heliotropine] | 120-57-0 | 0.1 | 5 |
| | Geraniol Fine FCC | 106-24-1 | 0.1 | 5 |
| Blend 30 | D-Limonene | 5989-27-5 | 15 | 85 |
| | Thyme Oil White | 8007-46-3 | 0.1 | 15 |
| | Methyl Salicylate | | 35 | 99 |
| Blend 31 | Thyme Oil White | 8007-46-3 | 0.1 | 5 |
| | Wintergreen Oil | 68917-75-9 | 0.1 | 8 |
| | Isopropyl myristate | 110-27-0 | 0.1 | 6 |
| | Span 80 | 1338-43-8 | 0.1 | 2 |
| | Isopar M | 64742-47-8 | 8 | 45 |
| | Water | 7732-18-5 | 40 | 99 |
| | Bifenthrin | 83657-04-3 | 0.005 | 0.2 |
| Blend 32 | Castor Oil hydrogenated - PEO40 | | 30 | 99 |
| | Lemon Grass Oil - India | | 10 | 70 |
| | Blend 1 | | 10 | 70 |
| Blend 33 | LFO | | 8 | 50 |
| | D-Limonene | 5989-27-5 | 35 | 99 |
| | Thyme Oil White | 8007-46-3 | 6 | 35 |
| | BSO | 977017-84-7 | 0.1 | 15 |
| Blend 34 | D-Limonene | 5989-27-5 | 0.1 | 25 |
| | Thyme Oil White | 8007-46-3 | 0.1 | 30 |
| | Blend 65 | | 30 | 99 |
| | Linalool Coeur | 78-70-6 | 0.1 | 5 |
| | Tetrahydrolinalool | 78-69-3 | 0.1 | 8 |
| | Vanillin | 121-33-5 | 0.06 | 0.5 |
| | Isopropyl myristate | 110-27-0 | 0.1 | 8 |
| | Piperonal (aldehyde) [Heliotropine] | 120-57-0 | 0.1 | 8 |
| | Geraniol Fine FCC | 106-24-1 | 0.1 | 4 |
| | Triethyl Citrate | 77-93-0 | 0.1 | 8 |
| | Isopar M | 64742-47-8 | 8 | 40 |
| Blend 35 | Isopropyl myristate | 110-27-0 | 20 | 99 |
| | Wintergreen Oil | | 25 | 99 |
| | Blend 68 | | 10 | 60 |
| Blend 36 | Wintergreen Oil | 68917-75-9 | 25 | 99 |
| | Isopropyl myristate | 110-27-0 | 20 | 99 |
| | Thyme Oil Red | 8007-46-3 | 10 | 60 |
| Blend 37 | Wintergreen Oil | 68917-75-9 | 25 | 99 |
| | Vanillin | 121-33-5 | 0.06 | 0.3 |
| | Isopropyl myristate | 110-27-0 | 20 | 99 |
| | Thyme Oil Red | 8007-46-3 | 10 | 60 |
| Blend 38 | Thyme Oil White | 8007-46-3 | 15 | 95 |
| | Isopropyl myristate | 110-27-0 | 25 | 99 |
| | Geraniol Fine FCC | 106-24-1 | 10 | 70 |
| Blend 39 | Isopropyl myristate | 110-27-0 | 25 | 99 |
| | Geraniol Fine FCC | 106-24-1 | 10 | 70 |
| | Blend 68 | | 20 | 99 |
| Blend 40 | Orange Terpenes | 68647-72-3 | 0.1 | 25 |
| | Blend 68 | | 0.1 | 30 |
| | Blend 69 | | 35 | 99 |
| | Blend 71 | | 6 | 40 |
| Blend 41 | Linalool Coeur | 78-70-6 | 10 | 70 |
| | Amyl Butyrate | 540-18-1 | 10 | 70 |
| | Anise Star Oil | | 30 | 99 |
| Blend 42 | Thyme Oil White | 8007-46-3 | 15 | 75 |
| | Amyl Butyrate | 540-18-1 | 10 | 70 |
| | Anise Star Oil | | 30 | 99 |
| Blend 43 | Tetrahydrolinalool | 78-69-3 | 10 | 70 |
| | Vanillin | 121-33-5 | 0.1 | 4 |
| | Hercolyn D | 8050-15-5 | 0.1 | 15 |
| | Isopropyl myristate | 110-27-0 | 8 | 45 |
| | Piperonal (aldehyde) [Heliotropine] | 120-57-0 | 0.1 | 25 |
| | Ethyl Linalool | 10339-55-6 | 10 | 70 |
| | Hedione | 24851-98-7 | 0.1 | 20 |
| | Triethyl Citrate | 77-93-0 | 5 | 30 |
| | Dipropylene glycol (DPG) | 246-770-3 | 0.1 | 25 |
| Blend 44 | Blend 63 | | 25 | 99 |
| | Thyme Oil White | | 30 | 99 |
| Blend 45 | Linalool coeur | 78-70-6 | 0.1 | 20 |
| | Tetrahydrolinalool | 78-69-3 | 0.1 | 25 |
| | Vanillin | 121-33-5 | 0.1 | 2 |
| | Isopropyl myristate | 110-27-0 | 0.1 | 30 |
| | Piperonal (aldehyde) [Heliotropine] | 120-57-0 | 0.1 | 30 |
| | Geraniol Fine FCC | 106-24-1 | 0.1 | 15 |
| | Triethyl citrate | 77-93-0 | 0.1 | 30 |
| | Thyme Oil White | | 30 | 99 |
| Blend 46 | Phenyl Ethyl Propionate | | 10 | 55 |
| | Benzyl Alcohol | 100-51-6 | 30 | 99 |
| | Methyl Salicylate | | 10 | 55 |
| | Blend 43 | | 8 | 40 |
| Blend 47 | Thyme Oil White | 8007-46-3 | 15 | 75 |
| | Amyl Butyrate | 540-18-1 | 10 | 70 |
| | Anise Star Oil | | 30 | 99 |
| | Genistein | | 0.005 | 0.1 |
| Blend 48 | Linalool coeur | 78-70-6 | 10 | 70 |
| | Amyl Butyrate | 540-18-1 | 10 | 70 |
| | Anise Star Oil | | 30 | 99 |
| | Thyme Oil White | | 0.005 | 0.1 |
| Blend 49 | LFO | | 10 | 70 |
| | BSO | 977017-84-7 | 10 | 70 |
| | Benzyl Alcohol | 100-51-6 | 30 | 99 |
| Blend 50 | Isopropyl myristate | 110-27-0 | 10 | 70 |
| | Wintergreen oil | | 15 | 90 |
| | Thyme oil white | | 8 | 40 |
| | Myristicin | | 15 | 99 |
| Blend 51 | Isopropyl myristate | 110-27-0 | 15 | 80 |
| | Wintergreen oil | | 15 | 95 |
| | Isopropyl alcohol | 67-63-0 | 0.1 | 10 |
| | Thyme oil white | | 8 | 40 |
| | Myristicin | | 15 | 75 |
| Blend 52 | Isopropyl myristate | 110-27-0 | 20 | 99 |
| | Wintergreen oil | | 25 | 99 |
| | Thyme oil white | | 10 | 60 |
| | Genistein | | 0.001 | 0.1 |
| Blend 53 | Isopropyl myristate | 110-27-0 | 20 | 99 |
| | Wintergreen oil | | 20 | 99 |
| | Isopropyl alcohol | 67-63-0 | 5 | 30 |
| | Thyme oil white | | 8 | 50 |
| | Genistein | | 0.001 | 0.1 |
| Blend 54 | Isopropyl myristate | 110-27-0 | 10 | 70 |
| | Wintergreen oil | | 15 | 90 |
| | Thyme oil white | | 8 | 40 |
| | Genistein | | 0.001 | 0.1 |
| | Myristicin | | 15 | 99 |
| Blend 55 | Mineral oil white | 8042-47-5 | 20 | 99 |
| | Wintergreen oil | | 25 | 99 |
| | Thyme oil white | | 10 | 60 |
| Blend 56 | Mineral oil white | 8042-47-5 | 10 | 50 |
| | Wintergreen oil | | 10 | 65 |
| | Thyme oil white | | 5 | 30 |
| | Benzaldehyde | | 30 | 99 |
| Blend 57 | Mineral oil white | 8042-47-5 | 10 | 55 |
| | Wintergreen oil | | 10 | 65 |
| | Thyme oil white | | 5 | 30 |
| | Genistein | | 15 | 75 |
| | Benzaldehyde | | 15 | 80 |
| Blend 58 | Linalool Coeur | 78-70-6 | 4 | 65 |
| | Thymol (crystal) | 89-83-8 | 20 | 99 |
| | Alpha-Pinene, 98% | 80-56-8 | 1 | 10 |
| | Para-Cymene | 99-87-6 | 1 | 55 |
| | Trans-Anethole | 4180-23-8 | 10 | 55 |
| Blend 59 | Linalool Coeur | 78-70-6 | 0.1 | 30 |
| | Thymol (crystal) | 89-83-8 | 25 | 99 |
| | Alpha-Pinene, 98% | 80-56-8 | 0.1 | 30 |
| | Para-Cymene | 99-87-6 | 15 | 99 |
| Blend 60 | Soy Bean Oil | 8016-70-4 | 15 | 75 |
| | Alpha-Pinene, 98% | 80-56-8 | 0.1 | 10 |
| | Para-Cymene | 99-87-6 | 15 | 85 |
| | Linalyl Acetate | 115-95-7 | 0.1 | 20 |
| | Thymol acetate | 528-79-0 | 20 | 99 |
| Blend 61 | Alpha-Pinene, 98% | 80-56-8 | 0.1 | 30 |
| | Para-Cymene | 99-87-6 | 10 | 55 |
| | Linalyl Acetate | 115-95-7 | 10 | 70 |
| | Thymol acetate | 528-79-0 | 30 | 99 |
| Blend 62 | Linalool Coeur | 78-70-6 | 10 | 60 |
| | Tetrahydrolinalool | 78-69-3 | 10 | 70 |
| | Vanillin | 121-33-5 | 0.1 | 8 |
| | Isopropyl myristate | 110-27-0 | 15 | 90 |
| | Piperonal (aldehyde) [Heliotropine] | 120-57-0 | 5 | 30 |
| | Geraniol Fine FCC | 106-24-1 | 8 | 40 |
| Blend 63 | Linalool Coeur | 78-70-6 | 8 | 40 |
| | Tetrahydrolinalool | 78-69-3 | 10 | 55 |
| | Vanillin | 121-33-5 | 0.1 | 4 |
| | Isopropyl myristate | 110-27-0 | 10 | 55 |
| | Piperonal (aldehyde) [Heliotropine] | 120-57-0 | 10 | 55 |
| | Geraniol Fine FCC | 106-24-1 | 5 | 30 |
| | Triethyl Citrate | 77-93-0 | 10 | 55 |
| Blend 64 | Linalool Coeur | 78-70-6 | 10 | 60 |
| | Tetrahydrolinalool | 78-69-3 | 10 | 70 |
| | Vanillin | 121-33-5 | 0.1 | 4 |
| | Isopropyl myristate | 110-27-0 | 10 | 70 |
| | Piperonal (aldehyde) [Heliotropine] | 120-57-0 | 10 | 70 |
| | Piperonyl Alcohol | 495-76-1 | 0.1 | 30 |
| Blend 65 | D-Limonene | 5989-27-5 | 25 | 99 |
| | Linalool Coeur | 78-70-6 | 0.1 | 4 |
| | Citral | 5392-40-5 | 5 | 30 |
| | gamma-terpinene | 99-85-4 | 5 | 30 |
| | Alpha-Pinene, 98% | 80-56-8 | 0.1 | 6 |
| | alpha-Terpineol | 98-55-5 | 0.1 | 20 |
| | Terpinolene | 586-62-9 | 0.1 | 20 |
| | Para-Cymene | 99-87-6 | 0.1 | 5 |
| | Linalyl Acetate | 115-95-7 | 0.1 | 8 |
| | Beta Pinene | 127-91-3 | 0.1 | 10 |
| | Camphor Dextro | 464-49-3 | 0.06 | 0.3 |
| | Terpinene 4 OL | 562-74-3 | 0.06 | 0.3 |
| | Alpha Terpinene | 99-86-5 | 0.1 | 10 |
| | Borneol L | 507-70-0 | 0.1 | 5 |
| | Camphene | 79-92-5 | 0.1 | 2 |
| | Decanal | 112-31-2 | 0.08 | 0.6 |
| | Dodecanal | 112-54-9 | 0.06 | 0.3 |
| | Fenchol Alpha | 512-13-0 | 0.001 | 0.1 |
| | Geranyl Acetate | 105-87-3 | 0.08 | 0.6 |
| | Isoborneol | 124-76-5 | 0.1 | 2 |
| | 2-Methyl 1,3-cyclohexadiene | 30640-46-1, 1888- | 0.1 | 2 |
| | | 90-0 | | |
| | Myrcene | 123-35-3 | 0.1 | 4 |
| | Nonanal | 124-19-6 | 0.001 | 0.1 |
| | Octanal | 124-13-0 | 0.05 | 0.2 |
| | Tocopherol Gamma (TENOX ®) | 54-28-4 | 0.001 | 0.1 |
| Blend 66 | D-Limonene | 5989-27-5 | 30 | 99 |
| | Linalool Coeur | 78-70-6 | 0.1 | 5 |
| | gamma-terpinene | 99-85-4 | 6 | 40 |
| | Alpha-Pinene, 98% | 80-56-8 | 0.1 | 8 |
| | Terpinolene | 586-62-9 | 0.1 | 25 |
| | Para-Cymene | 99-87-6 | 0.1 | 6 |
| | Linalyl Acetate | 115-95-7 | 0.1 | 10 |
| | Beta Pinene | 127-91-3 | 0.1 | 10 |
| | Camphor Dextro | 464-49-3 | 0.1 | 10 |
| | Terpinene 4 OL | 562-74-3 | 0.06 | 0.3 |
| | Alpha Terpinene | 99-86-5 | 0.08 | 0.6 |
| | Borneol L | 507-70-0 | 0.1 | 5 |
| | Camphene | 79-92-5 | 0.1 | 3 |
| | Decanal | 112-31-2 | 0.08 | 0.6 |
| | Dodecanal | 112-54-9 | 0.08 | 0.6 |
| | Fenchol Alpha | 512-13-0 | 0.001 | 0.1 |
| | Geranyl Acetate | 105-87-3 | 0.08 | 0.6 |
| | Isoborneol | 124-76-5 | 0.1 | 2 |
| | 2-Methyl 1,3-cyclohexadiene | 30640-46-1, 1888- | 0.1 | 2 |
| | | 90-0 | | |
| | Myrcene | 123-35-3 | 0.1 | 5 |
| | Nonanal | 124-19-6 | 0.001 | 0.2 |
| | Octanal | 124-13-0 | 0.05 | 0.3 |
| | Tocopherol Gamma (TENOX ®) | 54-28-4 | 0.001 | 0.2 |
| Blend 67 | D-Limonene | 5989-27-5 | 20 | 99 |
| | Linalool Coeur | 78-70-6 | 5 | 30 |
| | Alpha-Pinene, 98% | 80-56-8 | 0.1 | 15 |
| | Terpinolene | 586-62-9 | 5 | 30 |
| | Para-Cymene | 99-87-6 | 5 | 30 |
| | Linalyl Acetate | 115-95-7 | 0.1 | 15 |
| | Beta Pinene | 127-91-3 | 0.1 | 15 |
| | Alpha Terpinene | 99-86-5 | 0.1 | 15 |
| | Camphene | 79-92-5 | 0.1 | 20 |
| | Myrcene | 123-35-3 | 0.1 | 30 |
| Blend 68 | D-Limonene | 5989-27-5 | 0.08 | 1 |
| | Thyme Oil Red | 8007-46-3 | 0.1 | 4 |
| | Thymol (crystal) | 89-83-8 | 30 | 99 |
| | alpha-Terpineol | 98-55-5 | 0.1 | 6 |
| | Para-Cymene | 99-87-6 | 10 | 60 |
| | Linalyl Acetate | 115-95-7 | 0.1 | 5 |
| | Caryophyllene-B | 87-44-5 | 0.1 | 10 |
| | Borneol L | 507-70-0 | 0.1 | 6 |
| | Myrcene | 123-35-3 | 0.1 | 4 |
| | Tea Tree Oil | | 0.1 | 6 |
| | Cypress Oil | | 0.1 | 10 |
| | Peppermint Terpenes | 8006-90-4 | 0.1 | 30 |
| | Linalool 90 | | 0.1 | 10 |
| Blend 69 | D-Limonene | 5989-27-5 | 30 | 99 |
| | Citral | 5392-40-5 | 0.1 | 25 |
| | gamma-terpinene | 99-85-4 | 5 | 30 |
| | Alpha-Pinene, 98% | 80-56-8 | 0.1 | 5 |
| | alpha-Terpineol | 98-55-5 | 0.1 | 15 |
| | Terpinolene | 586-62-9 | 0.1 | 20 |
| | Lime Distilled Oil | | 0.06 | 0.3 |
| | Lime Expressed Oil | | 0.06 | 0.3 |
| | Linalyl Acetate | 115-95-7 | 0.1 | 6 |
| | Caryophyllene-B | 87-44-5 | 0.06 | 0.3 |
| | Beta Pinene | 127-91-3 | 0.1 | 8 |
| | Terpinene 4 OL | 562-74-3 | 0.005 | 0.2 |
| | Alpha Terpinene | 99-86-5 | 0.1 | 6 |
| | Borneol L | 507-70-0 | 0.1 | 5 |
| | Camphene | 79-92-5 | 0.1 | 2 |
| | Geranyl Acetate | 105-87-3 | 0.08 | 0.6 |
| | Isoborneol | 124-76-5 | 0.06 | 0.3 |
| | Linalool 90 | | 0.1 | 3 |
| | Camphor Gum | | 0.005 | 0.2 |
| | Aldehyde C-10 | | 0.005 | 0.2 |
| | Aldehyde C-12 | | 0.06 | 0.3 |
| Blend 70 | Eugenol | 97-53-0 | 0.003 | 0.1 |
| | Eucalyptol (1,8 Cineole) | | 0.05 | 0.3 |
| | Methyl Salicylate | | 60 | 99.9 |
| | Linalool 90 | | 0.05 | 0.3 |
| | Ethyl Salicylate | | 0.05 | 0.3 |
| Blend 71 | Tetrahydrolinalool | 78-69-3 | 6 | 35 |
| | Hercolyn D | 8050-15-5 | 0.1 | 25 |
| | Isopropyl myristate | 110-27-0 | 0.1 | 20 |
| | Piperonal (aldehyde) [Heliotropine] | 120-57-0 | 5 | 30 |
| | Ethyl Linalool | 10339-55-6 | 5 | 30 |
| | Triethyl Citrate | 77-93-0 | 0.1 | 30 |
| | Dipropylene glycol (DPG) | 246-770-3 | 5 | 30 |
| | Cinnamic Alcohol | 104-54-1 | 0.1 | 5 |
| | Eugenol | 97-53-0 | 0.1 | 5 |
| | Phenyl Ethyl Alcohol | 60-12-8 | 10 | 65 |
| | Iso Eugenol | | 0.08 | 1 |
| | Methyl Dihydrojasmonate | | 5 | 30 |
| Blend 72 | Linalool Coeur | 78-70-6 | 8 | 40 |
| | Tetrahydrolinalool | 78-69-3 | 10 | 70 |
| | Vanillin | 121-33-5 | 0.1 | 8 |
| | Isopropyl myristate | 110-27-0 | 15 | 85 |
| | Piperonal (aldehyde) [Heliotropine] | 120-57-0 | 5 | 30 |
| | Piperonyl Alcohol | 495-76-1 | 5 | 30 |
| | Geraniol Fine FCC | 106-24-1 | 5 | 30 |
| Blend 73 | Blend 11 | | 50 | 99 |
| | Stock 10% SLS Solution | | 5 | 30 |
| Blend 74 | Polyglycerol-4-oleate | 9007-48-1 | 0.1 | 3 |
| | Lecithin | 8002-43-5 | 0.08 | 0.6 |
| | Water | 7732-18-5 | 5 | 30 |
| | Blend 11 | | 50 | 99 |
| Blend 75 | Potassium Sorbate | 590-00-1 or 24634- | 0.1 | 4 |
| | | 61-5 | | |
| | Xanthan Gum | 11138-66-2 | 0.08 | 1 |
| | Water | 7732-18-5 | 45 | 99 |
| | Blend 74 | | 10 | 50 |
| Blend 76 | Potassium Sorbate | 590-00-1 or 24634- | 0.1 | 2 |
| | | 61-5 | | |
| | Polyglycerol-4-oleate | 9007-48-1 | 0.1 | 2 |
| | Xanthan Gum | 11138-66-2 | 0.08 | 1 |
| | Lecithin | 8002-43-5 | 0.06 | 0.3 |
| | Water | 7732-18-5 | 20 | 99 |
| | Blend 11 | | 15 | 99 |
| Blend 77 | Thyme Oil White | 8007-46-3 | 0.1 | 25 |
| | Wintergreen Oil | 68917-75-9 | 2 | 55 |
| | Isopropyl myristate | 110-27-0 | 1 | 40 |
| | Potassium Sorbate | 590-00-1 or 24634- | 0.06 | 0.3 |
| | | 61-5 | | |
| | Polyglycerol-4-oleate | 9007-48-1 | 0.1 | 2 |
| | Xanthan Gum | 11138-66-2 | 0.08 | 1 |
| | Lecithin | 8002-43-5 | 0.06 | 0.3 |
| | Water | 7732-18-5 | 20 | 99 |
| Blend 78 | Polyglycerol-4-oleate | 9007-48-1 | 0.1 | 3 |
| | Lecithin | 8002-43-5 | 0.08 | 0.6 |
| | Water | 7732-18-5 | 5 | 30 |
| | Blend 11 | | 50 | 99 |
| Blend 79 | Water | 7732-18-5 | 0.1 | 20 |
| | Blend 74 | | 40 | 99 |
| | Stock 2.5% Xanthan-1% Ksorbate | | 6 | 40 |
| Blend 80 | Water | 7732-18-5 | 0.1 | 10 |
| | Blend 78 | | 45 | 99 |
| | Stock 2.5% Xanthan-1% Ksorbate | | 6 | 40 |
| Blend 81 | Potassium Sorbate | 590-00-1 or 24634- | 0.1 | 4 |
| | | 61-5 | | |
| | Xanthan Gum | 11138-66-2 | 0.08 | 1 |
| | Water | 7732-18-5 | 45 | 99 |
| | Blend 78 | | 10 | 50 |
| Blend 82 | Blend 1 | | 0.1 | 8 |
| | Water | | 60 | 99 |
| Blend 83 | Polyglycerol-4-oleate | 9007-48-1 | 0.1 | 3 |
| | Lecithin | 8002-43-5 | 0.08 | 0.6 |
| | Water | 7732-18-5 | 5 | 30 |
| | Blend 11 | | 50 | 99 |
| Blend 84 | Potassium Sorbate | 590-00-1 or 24634- | 0.1 | 4 |
| | | 61-5 | | |
| | Xanthan Gum | 11138-66-2 | 0.08 | 1 |
| | Water | 7732-18-5 | 45 | 99 |
| | Blend 83 | | 10 | 50 |
| Blend 85 | Citronella Oil | 106-22-9 | 0.08 | 0.6 |
| | Carbopol 940 | [9003-01-4] | 0.08 | 0.6 |
| | BHT (butylated hydroxytoluene) | 128-37-0 | 0.06 | 0.3 |
| | Water | 7732-18-5 | 30 | 99 |
| | Emulsifying Wax | 67762-27-0, 9005- | 8 | 40 |
| | | 67-8 | | |
| | Light Liquid Paraffin | 8012-95-1 | 0.1 | 10 |
| | White Soft Paraffin | [8009-03-8] | 0.1 | 25 |
| | Sodium Metabisulphate | [7681-57-4] | 0.08 | 1 |
| | Propylene Glycol | [57-55-6] | 0.1 | 6 |
| | Methyl Paraben | [99-76-3] | 0.08 | 0.6 |
| | Propyl Paraben | [94-13-3] | 0.005 | 0.2 |
| | Cresmer RH40 hydrogenated castor oil | [61791-12-6] | 0.1 | 15 |
| | Triethanolamine | [102-71-6] | 0.08 | 0.6 |
| | Vitamin E Acetate | [58-95-7] | 0.002 | 0.08 |
| | Disodium EDTA | [139-33-3] | 0.005 | 0.2 |
| | Blend 1 | | 0.1 | 15 |
| Blend 86 | Span 80 | 1338-43-8 | 0.005 | 0.2 |
| | Sodium Benzoate | 532-32-1 | 0.08 | 0.6 |
| | Isopar M | 64742-47-8 | 15 | 85 |
| | A46 Propellant | | 8 | 45 |
| | Water | 7732-18-5 | 25 | 99 |
| | Isopropyl alcohol | 67-63-0 | 0.1 | 5 |
| | Blend 8 | | 6 | 40 |
| Blend 87 | Isopar M | 64742-47-8 | 30 | 99 |
| | A46 Propellant | | 20 | 99 |
| | Isopropyl alcohol | 67-63-0 | 0.1 | 10 |
| | Blend 25 | | 0.1 | 20 |
| Blend 88 | Isopar M | 64742-47-8 | 30 | 99 |
| | A46 Propellant | | 20 | 99 |
| | Bifenthrin | 83657-04-3 | 0.005 | 0.2 |
| | Isopropyl alcohol | 67-63-0 | 0.1 | 10 |
| | Blend 25 | | 0.1 | 20 |
| Blend 89 | Isopar M | 64742-47-8 | 30 | 99 |
| | A46 Propellant | | 20 | 99 |
| | Blend 20 | | 0.1 | 20 |
| Blend 90 | Potassium Sorbate | 590-00-1 or 24634- | 0.06 | 0.3 |
| | | 61-5 | | |
| | Polyglycerol-4-oleate | 9007-48-1 | 0.08 | 0.6 |
| | Xanthan Gum | 11138-66-2 | 0.08 | 0.6 |
| | Lecithin | 8002-43-5 | 0.003 | 0.1 |
| | Water | 7732-18-5 | 45 | 99 |
| | Isopropyl alcohol | 67-63-0 | 0.1 | 8 |
| | Blend 35 | | 8 | 45 |
| Blend 91 | Potassium Sorbate | 590-00-1 or 24634- | 0.06 | 0.3 |
| | | 61-5 | | |
| | Polyglycerol-4-oleate | 9007-48-1 | 0.08 | 0.6 |
| | Xanthan Gum | 11138-66-2 | 0.08 | 1 |
| | Lecithin | 8002-43-5 | 0.003 | 0.1 |
| | Water | 7732-18-5 | 50 | 99 |
| | Blend 35 | | 8 | 40 |
| Blend 92 | Isopropyl myristate | 110-27-0 | 0.1 | 10 |
| | Geraniol Fine FCC | 106-24-1 | 0.1 | 8 |
| | Potassium Sorbate | 590-00-1 or 24634- | 0.06 | 0.3 |
| | | 61-5 | | |
| | Polyglycerol-4-oleate | 9007-48-1 | 0.1 | 2 |
| | Xanthan Gum | 11138-66-2 | 0.08 | 1 |
| | Lecithin | 8002-43-5 | 0.05 | 0.2 |
| | Water | 7732-18-5 | 50 | 99 |
| | Blend 68 | | 0.1 | 10 |
| | Isopropyl alcohol | 67-63-0 | 0.1 | 8 |
| Blend 93 | Wintergreen Oil | 68917-75-9 | 0.1 | 15 |
| | Isopropyl myristate | 110-27-0 | 0.1 | 10 |
| | Thyme Oil Red | 8007-46-3 | 0.1 | 6 |
| | Stock 0.3% SLS-0.1% Xanthan Soln | | 55 | 99 |
| Blend 94 | Stock 0.3% SLS & 0.1% Xanthan Soln | | 60 | 99 |
| | Blend 38 | | 0.1 | 15 |
| Blend 95 | Lecithin, Soya | 8030-76-0 | 0.08 | 0.6 |
| | Polyglycerol-4-oleate | 9007-48-1 | 0.1 | 3 |
| | Water | 7732-18-5 | 5 | 30 |
| | Blend 11 | | 50 | 99 |
| Blend 96 | Thyme Oil White | 8007-46-3 | 20 | 99 |
| | Isopropyl myristate | 110-27-0 | 15 | 95 |
| | Lecithin, Soya | 8030-76-0 | 0.08 | 0.6 |
| | Polyglycerol-4-oleate | 9007-48-1 | 0.1 | 3 |
| | Water | 7732-18-5 | 5 | 30 |
| | Wintergreen Oil | | 10 | 65 |
| Blend 97 | Lecithin, Soya | 8030-76-0 | 0.06 | 0.3 |
| | Polyglycerol-4-oleate | 9007-48-1 | 0.1 | 3 |
| | Water | 7732-18-5 | 5 | 30 |
| | Blend 7 | | 50 | 99 |
| Blend 98 | Thyme Oil White | 8007-46-3 | 10 | 55 |
| | Wintergreen Oil | 68917-75-9 | 20 | 99 |
| | Vanillin | 121-33-5 | 0.1 | 4 |
| | Isopropyl myristate | 110-27-0 | 15 | 90 |
| | Lecithin, Soya | 8030-76-0 | 0.06 | 0.3 |
| | Polyglycerol-4-oleate | 9007-48-1 | 0.1 | 3 |
| | Water | 7732-18-5 | 5 | 30 |
| Blend 99 | Polyglycerol-4-oleate | 9007-48-1 | 0.1 | 6 |
| | Water | 7732-18-5 | 0.1 | 25 |
| | Blend 11 | | 50 | 99 |
| Blend 100 | Thyme Oil White | 8007-46-3 | 20 | 99 |
| | Isopropyl myristate | 110-27-0 | 15 | 95 |
| | Polyglycerol-4-oleate | 9007-48-1 | 0.1 | 6 |
| | Water | 7732-18-5 | 0.1 | 25 |
| | Wintergreen Oil | | 10 | 65 |
| Blend 101 | Potassium Sorbate | 590-00-1 or 24634- | 0.06 | 0.3 |
| | | 61-5 | | |
| | Polyglycerol-4-oleate | 9007-48-1 | 0.1 | 6 |
| | Xanthan Gum | 11138-66-2 | 0.08 | 1 |
| | Water | 7732-18-5 | 50 | 99 |
| | Blend 97 | | 6 | 35 |
| Blend 102 | D-Limonene | 5989-27-5 | 0.1 | 15 |
| | Thyme Oil White | 8007-46-3 | 0.1 | 5 |
| | Lecithin, Soya | 8030-76-0 | 0.001 | 0.04 |
| | Potassium Sorbate | 590-00-1 or 24634- | 0.06 | 0.3 |
| | | 61-5 | | |
| | Polyglycerol-4-oleate | 9007-48-1 | 0.1 | 6 |
| | Xanthan Gum | 11138-66-2 | 0.08 | 1 |
| | Water | 7732-18-5 | 50 | 99 |
| | Wintergreen Oil | | 0.1 | 10 |
| Blend 103 | Potassium Sorbate | 590-00-1 or 24634- | 0.06 | 0.3 |
| | | 61-5 | | |
| | Xanthan Gum | 11138-66-2 | 0.08 | 1 |
| | Water | 7732-18-5 | 50 | 99 |
| | Blend 95 | | 6 | 35 |
| Blend 104 | Thyme Oil White | 8007-46-3 | 0.1 | 10 |
| | Isopropyl myristate | 110-27-0 | 0.1 | 10 |
| | Lecithin, Soya | 8030-76-0 | 0.002 | 0.08 |
| | Potassium Sorbate | 590-00-1 or 24634- | 0.06 | 0.3 |
| | | 61-5 | | |
| | Polyglycerol-4-oleate | 9007-48-1 | 0.06 | 0.3 |
| | Xanthan Gum | 11138-66-2 | 0.08 | 1 |
| | Water | 7732-18-5 | 55 | 99 |
| | Wintergreen Oil | | 0.1 | 8 |
| Blend 105 | Potassium Sorbate | 590-00-1 or 24634- | 0.06 | 0.3 |
| | | 61-5 | | |
| | Xanthan Gum | 11138-66-2 | 0.08 | 1 |
| | Water | 7732-18-5 | 50 | 99 |
| | Blend 99 | | 6 | 35 |
| Blend 106 | Thyme Oil White | 8007-46-3 | 0.1 | 10 |
| | Wintergreen Oil | 68917-75-9 | 0.1 | 8 |
| | Isopropyl myristate | 110-27-0 | 0.1 | 10 |
| | Potassium Sorbate | 590-00-1 or 24634- | 0.06 | 0.3 |
| | | 61-5 | | |
| | Polyglycerol-4-oleate | 9007-48-1 | 0.08 | 0.6 |
| | Xanthan Gum | 11138-66-2 | 0.08 | 1 |
| | Water | 7732-18-5 | 55 | 99 |
| Blend 107 | Potassium Sorbate | 590-00-1 or 24634- | 0.1 | 4 |
| | | 61-5 | | |
| | Xanthan Gum | 11138-66-2 | 0.1 | 8 |
| | Water | 7732-18-5 | 60 | 99 |
| Blend 108 | Sodium Benzoate | 532-32-1 | 0.1 | 6 |
| | Water | 7732-18-5 | 60 | 99 |
| Blend 109 | Span 80 | 1338-43-8 | 0.1 | 4 |
| | Tween 80 | | 0.1 | 5 |
| | Isopar M | 64742-47-8 | 8 | 40 |
| | Water | 7732-18-5 | 35 | 99 |
| | Blend 8 | | 0.1 | 10 |
| | 2% Sodium Benzoate | | 6 | 35 |
| Blend 110 | D-Limonene | 5989-27-5 | 0.1 | 5 |
| | Thyme Oil White | 8007-46-3 | 0.1 | 2 |
| | Wintergreen Oil | 68917-75-9 | 0.1 | 3 |
| | Span 80 | 1338-43-8 | 0.1 | 4 |
| | Tween 80 | | 0.1 | 5 |
| | Sodium Benzoate | 532-32-1 | 0.08 | 0.6 |
| | Isopar M | 64742-47-8 | 8 | 40 |
| | Water | 7732-18-5 | 40 | 99 |
| Blend 111 | Propellent A70 | | 10 | 65 |
| | Blend 109 | | 45 | 99 |
| Blend 112 | D-Limonene | 5989-27-5 | 0.1 | 5 |
| | Thyme Oil White | 8007-46-3 | 0.08 | 1 |
| | Wintergreen Oil | 68917-75-9 | 0.1 | 3 |
| | Span 80 | 1338-43-8 | 0.1 | 3 |
| | Tween 80 | | 0.1 | 5 |
| | Sodium Benzoate | 532-32-1 | 0.08 | 0.6 |
| | Isopar M | 64742-47-8 | 6 | 35 |
| | Water | 7732-18-5 | 35 | 99 |
| | Propellent A70 | | 10 | 65 |
| Blend 113 | Sodium Lauryl Sulfate | 151-21-3 | 5 | 30 |
| | Water | 7732-18-5 | 55 | 99 |
| Blend 114 | Sodium Lauryl Sulfate | 151-21-3 | 0.08 | 1 |
| | Xanthan Gum | 11138-66-2 | 0.06 | 0.3 |
| | Water | 7732-18-5 | 60 | 99.9 |
| Blend 115 | Citronella Oil | 106-22-9 | 0.08 | 0.6 |
| | Carbopol 940 | [9003-01-4] | 0.08 | 0.6 |
| | BHT (butylated hydroxytoluene) | 128-37-0 | 0.06 | 0.3 |
| | Water | 7732-18-5 | 30 | 99 |
| | Emulsifying Wax | 67762-27-0, 9005- | 8 | 40 |
| | | 67-8 | | |
| | Light Liquid Paraffin | 8012-95-1 | 0.1 | 10 |
| | White Soft Paraffin | [8009-03-8] | 0.1 | 25 |
| | Sodium Metabisulphate | [7681-57-4] | 0.08 | 1 |
| | Propylene Glycol | [57-55-6] | 0.1 | 6 |
| | Cresmer RH40 hydrogenated castor oil | [61791-12-6] | 0.1 | 15 |
| | Triethanolamine | [102-71-6] | 0.08 | 0.6 |
| | Vitamin E Acetate | [58-95-7] | 0.002 | 0.08 |
| | Disodium EDTA | [139-33-3] | 0.005 | 0.2 |
| | Blend 1 | | 0.1 | 15 |
| Blend 116 | Water | 7732-18-5 | 20 | 99 |
| | Blend 75 | | 35 | 99 |
| Blend 117 | D-Limonene | 5989-27-5 | 0.1 | 10 |
| | Thyme Oil White | 8007-46-3 | 0.1 | 15 |
| | Benzyl Alcohol | 100-51-6 | 8 | 50 |
| | Isopar M | 64742-47-8 | 10 | 65 |
| | Water | 7732-18-5 | 25 | 99 |
| | Bifenthrin | 83657-04-3 | 0.005 | 0.2 |
| | Blend 63 | | 0.1 | 15 |
| | Stock 10% SLS Solution | | 0.1 | 10 |
| Blend 118 | Thyme Oil White | 8007-46-3 | 0.1 | 2 |
| | Wintergreen Oil | 68917-75-9 | 0.1 | 3 |
| | Isopropyl myristate | 110-27-0 | 0.1 | 3 |
| | Sodium Lauryl Sulfate | 151-21-3 | 0.002 | 0.08 |
| | Water | 7732-18-5 | 60 | 99 |
| Blend 119 | Thyme Oil White | 8007-46-3 | 0.1 | 4 |
| | Wintergreen Oil | 68917-75-9 | 0.1 | 8 |
| | Isopropyl myristate | 110-27-0 | 0.1 | 5 |
| | AgSorb clay carrier | | 60 | 99 |
| Blend 120 | Thyme Oil White | 8007-46-3 | 0.1 | 4 |
| | Wintergreen Oil | 68917-75-9 | 0.1 | 8 |
| | Isopropyl myristate | 110-27-0 | 0.1 | 5 |
| | DG Lite | | 60 | 99 |
| Blend 121 | D-Limonene | 5989-27-5 | 15 | 75 |
| | Thyme Oil White | 8007-46-3 | 0.1 | 4 |
| | Linalool Coeur | 78-70-6 | 0.08 | 0.6 |
| | Tetrahydrolinalool | 78-69-3 | 0.08 | 0.6 |
| | Vanillin | 121-33-5 | 0.002 | 0.08 |
| | Isopropyl myristate | 110-27-0 | 0.08 | 0.6 |
| | Piperonal (aldehyde) [Heliotropine] | 120-57-0 | 0.08 | 0.6 |
| | Blend 66 | | 0.1 | 10 |
| | Geraniol 60 | 106-24-1 | 0.06 | 0.3 |
| | Triethyl Citrate | 77-93-0 | 0.08 | 0.6 |
| | Water | 7732-18-5 | 35 | 99 |
| | Stock 10% SLS Solution | | 0.1 | 10 |
| Blend 122 | Miracle Gro (Sterile) | | 60 | 99 |
| | Blend 11 | | 0.1 | 15 |
| Blend 123 | Thyme Oil White | 8007-46-3 | 15 | 75 |
| | Amyl Butyrate | 540-18-1 | 15 | 75 |
| | Anise Star Oil | | 30 | 99 |
| | Genistein | | 0.001 | 0.1 |
| Blend 124 | Linalool Coeur | | 0.1 | 20 |
| | Tetrahydrolinalool | | 0.1 | 25 |
| | Vanillin | | 0.1 | 2 |
| | Isopropyl myristate | | 0.1 | 30 |
| | Piperonal (aldehyde) [Heliotropine] | | 0.1 | 30 |
| | Geraniol Fine FCC | | 0.1 | 15 |
| | Triethyl Citrate | | 0.1 | 30 |
| | Thyme Oil White | | 30 | 99 |
| Blend 125 | D-Limonene | 5989-27-5 | 5 | 30 |
| | Linalool Coeur | 78-70-6 | 8 | 40 |
| | Tetrahydrolinalool | 78-69-3 | 15 | 75 |
| | Vanillin | 121-33-5 | 0.1 | 8 |
| | Isopropyl myristate | 110-27-0 | 15 | 85 |
| | Piperonal (aldehyde) | 120-57-0 | 5 | 30 |
| | Geraniol 60 | | 5 | 30 |
| Blend 126 | D-Limonene | 5989-27-5 | 45 | 99 |
| | Thyme Oil White | 8007-46-3 | 0.1 | 10 |
| | Linalool Coeur | 78-70-6 | 0.1 | 2 |
| | Tetrahydrolinalool | 78-69-3 | 0.1 | 3 |
| | Vanillin | 121-33-5 | 0.005 | 0.2 |
| | Isopropyl myristate | 110-27-0 | 0.1 | 3 |
| | Piperonal (aldehyde) [Heliotropine] | 120-57-0 | 0.1 | 3 |
| | Blend 66 | | 5 | 30 |
| | Geraniol 60 | | 0.1 | 2 |
| | Triethyl Citrate | 77-93-0 | 0.1 | 3 |

The present invention comprises compositions for controlling insects and methods for using these compositions. The present invention comprises compositions for controlling insects, which comprise one or more plant essential oils and methods for using these compositions. The plant essential oils, when combined, can have a synergistic effect. The compositions of the present invention can include any of the following oils listed below, or mixtures thereof:

| | | |
|---|---|---|
| trans-anethole | lime oil | piperonal |
| Black seed oil (BSO) | d-limonene | piperonyl |
| camphene | linalyl anthranilate | piperonyl acetate |
| carvacrol | linalool | piperonyl alcohol |
| d-carvone | lindenol | piperonyl amine |
| l-carvone | methyl citrate | quinone |
| 1,8-cineole | methyl di-hydrojasmonate | sabinene |
| p-cymene | myrcene | α-terpinene |
| diethyl phthalate | perillyl alcohol | terpinene 900 |
| eugenol | phenyl acetaldehyde | α-terpineol |
| geraniol | phenylethyl alcohol | gamma-terpineol |
| isopropyl citrate | phenylethyl propionate | 2-tert-butyl-p- |
| lemon grass oil | α-pinene | quinone |
| lilac flower oil (LFO) | β-pinene | α-thujone |
| | | thyme oil |
| | | thymol |

The compositions of the present invention may also include any of the following oils listed below, or mixtures thereof:

Allyl sulfide
Allyl trisulfide
Allyl-disulfide
Anethole
Artemisia alcohol acetate
Benzyl acetate
Benzyl alcohol
Bergamotene
β-bisabolene
Bisabolene oxide
α-bisabolol
Bisabolol oxide
Bisobolol oxide β
Bornyl acetate
β-bourbonene
α-cadinol
Camphene
α-campholene
α-campholene aldehyde
camphor
carbaryl
Caryophyllene oxide
Chamazulene
Chrysanthemate ester
Chrysanthemic acid
Chrysanthemyl alcohol
Cinnamaldehyde
Cis-verbenol
Citral A
Citral B
Citronellal
Citronellol
Citronellyl acetate
Citronellyl formate
α-copaene
cornmint oil
β-costol
Cryptone
Curzerenone
d-Carvone
l-Carvone
Davanone
Diallyl tetrasulfide
dihydropyrocurzerenone
Dihydrotagentone
β-elemene
gamma-elemene
Elmol
Estragole
2-ethyl-2-hexen-1-ol
Eugenol acetate
α-farnesene
(Z,E)-α-farnesene
E-β-farnesene
Fenchone
Forskolin
Furanodiene
Furanoeudesma-1,3-diene
Furanoeudesma-1,4-diene
Furano germacra 1,10(15)-diene-6-one
Furanosesquiterpene
Geraniol
Geraniol acetate
Germacrene D
Germacrene B
α-gurjunene
α-humulene
α-ionone
β-ionone
Isoborneol
Isofuranogermacrene
Iso-menthone
Iso-pulegone
Jasmone
cis-jasmone
Lavandustin A
Lilac flower oil
Limonene
Linalool
Linalyl acetate
Lindestrene
Methyl-allyl-trisulfide
Menthol
2-methoxy furanodiene
menthone
Menthyl acetate
Methyl salicylate
Methyl cinnamate
Menthyl salicylate
Myrtenal
Neraldimethyl acetate
Nerolidol
Nonanone
1-octanol
E ocimenone
Z ocimenone
3-octanone
Ocimene
Octyl acetate
Peppermint oil
Permethrin
α-phellandrene
β-phellandrene
piperonal
Prenal
Propargite
Pulegone
Pyrethrum
Sabinene
Sabinyl acetate
α-santalene
Santalol
Sativen
δ-selinene
β-sesquiphellandrene
Spathulenol
Tagetone
Tamoxifen
Tebufenozide
α-terpinene
4-terpineol
α-terpinolene
α-terpinyl acetate
tetrahydrofurfuryl alcohol
α-thujene
Thymyl methyl ether
Trans-caryophyllene
Trans-pinocarveol
Trans-verbenol
Verbenone
Yomogi alcohol
Zingiberene

In those compositions including more than one oil, each oil can make up between about 1% to about 99%, by weight, of the composition mixture. For example, one composition of the present invention comprises about 1% thymol and about 99% geraniol. Optionally, the compositions can additionally comprise a fixed oil, which is a non-volatile non-scented plant oil. For example, the composition could include one or more of the following fixed oils listed below:

| | | |
|---|---|---|
| castor oil | mineral oil | safflower oil |
| corn oil | olive oil | sesame oil |
| cumin oil | peanut oil | soy bean oil |
| | | canola oil |

For example, one composition of the present invention includes about 1% thymol, about 50% geraniol and about 49% mineral oil. Additionally, it is contemplated that these compositions may be made up of generally regarded as safe (GRAS) compounds, for example: thyme oil, geraniol, lemon grass oil, lilac flower oil, black seed oil, lime oil, eugenol, castor oil, mineral oil, and safflower oil.

Embodiments of the invention can also include enzyme cascade inhibitors, such as, for example, PD98059, an inhibitor of MAPK extracellular signaling-regulated kinase (ERK) kinase (MEK).

Some embodiments of the invention can include dispersible granules, such as, for example, DG Lite™, and the like.

Some embodiments of the invention can include skin creams and formulations, such as, for example, CAR-01-097, Cresmer RH40 hydrogenated, and the like.

In certain embodiments wherein the composition includes Lilac Flower Oil (LFO), one or more of the following compounds can be substituted for the LFO: Tetrahydrolinalool, Ethyl Linalool, Heliotropine, Hedione, Hercolyn D, and Triethyl Citrate. In certain embodiments wherein the composition includes LFO, a blend of the following compounds can be substituted for the LFO: Isopropyl myristate, Tetrahydrolinalool FCC, Linalool, Geraniol Fine FCC, Piperonal (aldehyde), and Vanillin. In certain embodiments wherein the composition includes LFO, a blend of the following compounds can be substituted for the LFO: Isopropyl myristate, Tetrahydrolinalool, Linalool, Geraniol, Piperonal (aldehyde), Vanillin, Methyl Salicylate, and D-limonene.

In certain embodiments wherein the composition includes Black Seed Oil (BSO), one or more of the following compounds can be substituted for the BSO: α-thujene; α-pinene; β-pinene; p-cymene; limonene; and tert-butyl-p-benzoquinone.

In certain exemplary embodiments wherein the composition includes Thyme Oil, one or more of the following compounds can be substituted for the Thyme Oil: thymol, a-thujone, α-pinene, camphene, β-pinene, p-cymene, α-terpinene, linalool, borneol, β-caryophyllene, and carvacrol. Compounds used to prepare the exemplary compositions of the present invention can be obtained, for example, from the following sources: Millennium Chemicals, Inc. (Jacksonville, Fla.), Ungerer Company (Lincoln Park, N.J.), SAFC (Milwaukee, Wis.), and IFF Inc. (Hazlet, N.J.).

In some embodiments of the compositions, it can be desirable to include compounds each having a purity of about 60%, 65%, 70%, 75%, 80%, 85%, 90%, or 95%. For example, in some embodiments of the compositions that include geraniol, it can be desirable to include a geraniol that is at least about 60%, 85% or 95% pure. In some embodiments, it can be desirable to include a specific type of geraniol. For example, in some embodiments, the compositions can include: geraniol 60, geraniol 85, or geraniol 95. When geraniol is obtained as geraniol 60, geraniol 85, or geraniol 95, then forty percent, fifteen percent, or five percent of the oil can be Nerol. Nerol is a monoterpene (C₁₀H₁₈O), that can be extracted from attar of roses, oil of orange blossoms and oil of lavender. Embodiments of the present invention can include art-recognised ingredients normally used in such formulations. These ingredients can include, for example, antifoaming agents, antimicrobial agents, anti-oxidants, anti-redeposition agents, bleaches, colorants, emulsifiers, enzymes, fats, fluorescent materials, fungicides, hydrotropes, moisturisers, optical brighteners, perfume carriers, perfume, preservatives, proteins, silicones, soil release agents, solubilisers, sugar derivatives, sun screens, surfactants, vitamins waxes, and the like.

In certain embodiments, embodiments of the present invention can also contain other adjuvants or modifiers such as one or more therapeutically or cosmetically active ingredients. Exemplary therapeutic or cosmetically active ingredients useful in the compositions of the invention can include, for example, fungicides, sunscreening agents, sunblocking agents, vitamins, tanning agents, plant extracts, anti-inflammatory agents, anti-oxidants, radical scavenging agents, retinoids, alpha-hydroxy acids, emollients, antiseptics, antibiotics, antibacterial agents, antihistamines, and the like, and can be present in an amount effective for achieving the therapeutic or cosmetic result desired.

In some embodiments, compositions of this invention can include one or more materials that can function as an antioxidant, such as reducing agents and free radical scavengers. Suitable materials that can function as an antioxidant can include, for example: acetyl cysteine, ascorbic acid, t-butyl hydroquinone, cysteine, diamylhydroquinone, erythorbic acid, ferulic acid, hydroquinone, p-hydroxyanisole, hydroxylamine sulfate, magnesium ascorbate, magnesium ascorbyl phosphate, octocrylene, phloroglucinol, potassium ascorbyl tocopheryl phosphate, potassium sulfite, rutin, sodium ascorbate, sodium sulfite, sodium thloglycolate, thiodiglycol, thiodiglycolamide, thioglycolic acid, thiosalicylic acid, tocopherol, tocopheryl acetate, tocopheryl linoleate, tris(nonylphenyl)phosphite, and the like.

Embodiments of the invention can also include one or more materials that can function as a chelating agent to complex with metallic ions. This action can help to inactivate the metallic ions for the purpose of preventing their adverse effects on the stability or appearance of a formulated composition. Chelating agents suitable for use in an embodiment of this invention can include, for example, aminotrimethylene phosphonic acid, beta-alanine diacetic acid, calcium disodium EDTA, citric acid, cyclodextrin, cyclohexanediamine tetraacetic acid, diammonium citrate, diammonium EDTA, dipotassium EDTA, disodium azacycloheptane diphosphonate, disodium EDTA, disodium pyrophosphate, EDTA (ethylene diamine tetra acetic acid), gluconic acid, HEDTA (hydroxyethyl ethylene diamine triacetic acid), methyl cyclodextrin, pentapotassium triphosphate, pentasodium aminotrimethylene phosphonate, pentasodium triphosphate, pentetic acid, phytic acid, potassium citrate, potassium gluconate, sodium citrate, sodium diethylenetriamine pentamethylene phosphonate, sodium dihydroxyethylglycinate, sodium gluconate, sodium metaphosphate, sodium metasilicate, sodium phytate, triethanolamine (“TEA”)-EDTA, TEA-polyphosphate, tetrahydroxypropyl ethylenediamine, tetrapotassium pyrophosphate, tetrasodium EDTA, tetrasodium pyrophosphate, tripotassium EDTA, trisodium EDTA, trisodium HEDTA, trisodium phosphate, and the like.

Embodiments of the invention can also include one or more materials that can function as a humectant. A humectant is added to a composition to retard moisture loss during use, which effect is accomplished, in general, by the presence therein of hygroscopic materials.

The following table (Table 2) provides exemplary compositions of embodiments of the invention:

TABLE 2
Exemplary Compositions

| | | | | | | |
|---|---|---|---|---|---|---|
| Ingredients | Exemplified form | % Range 1 | % Range 2 | % Range 3 | % Range 4 | Exemplified % (w/w) |

Example 1 - Ingredient Family 1

| | | | | | | | | | | |
|---|---|---|---|---|---|---|---|---|---|---|
| Linalool | Linalool Coeur | 0.66% | 19.80% | 3.30% | 9.90% | 4.95% | 8.25% | 5.94% | 7.26% | 6.6 [symbol] |
| Base Oil | Soy Bean Oil | 2.40% | 72.00% | 12.00% | 36.00% | 18.00% | 30.00% | 21.60% | 26.40% | 24.0 [symbol] |
| Thymol | Thymol (crystal) | 3.72% | 99.00% | 18.60% | 55.80% | 27.90% | 46.50% | 33.48% | 40.92% | 37.2 [symbol] |
| Pinene | Alpha-Pinene, 98% | 0.38% | 11.40% | 1.90% | 5.70% | 2.85% | 4.75% | 3.42% | 4.18% | 3.80% |
| Cymene | Para-Cymene | 2.84% | 85.17% | 14.20% | 42.59% | 21.29% | 35.49% | 25.55% | 31.23% | 28.39% |

Example 2 - Ingredient Family 2

| | | | | | | | | | | |
|---|---|---|---|---|---|---|---|---|---|---|
| Thyme Oil | Thyme Oil White | 2.06% | 61.80% | 10.30% | 30.90% | 15.45% | 25.75% | 18.54% | 22.66% | 20.60% |
| Wintergreen Oil | Wintergreen Oil | 4.51% | 99.00% | 22.55% | 67.65% | 33.83% | 56.38% | 40.59% | 49.61% | 45.10% |
| Isopropyl myristate | Isopropyl myristate | 3.43% | 99.00% | 17.15% | 51.45% | 25.73% | 42.88% | 30.87% | 37.73% | 34.30% |

Example 3 - Ingredient Family 3

| | | | | | | | | | | |
|---|---|---|---|---|---|---|---|---|---|---|
| Thyme Oil | Thyme Oil White | 2.48% | 74.25% | 12.38% | 37.13% | 18.56% | 30.94% | 22.28% | 27.23% | 24.75% |
| Amyl Butyrate | Amyl Butyrate | 2.30% | 69.12% | 11.52% | 34.56% | 17.28% | 28.80% | 20.74% | 25.34% | 23.04% |
| Anise Star Oil | Anise Star Oil | 5.22% | 99.00% | 26.11% | 78.32% | 39.16% | 65.26% | 46.99% | 57.43% | 52.21% |

Example 4 - Ingredient Family 4

| | | | | | | | | | | |
|---|---|---|---|---|---|---|---|---|---|---|
| Thyme Oil | Thyme Oil White | 2.48% | 74.25% | 12.38% | 37.13% | 18.56% | 30.94% | 22.28% | 27.23% | 24.75% |
| Amyl Butyrate | Amyl Butyrate | 2.30% | 69.12% | 11.52% | 34.56% | 17.28% | 28.80% | 20.74% | 25.34% | 23.04% |
| Anise Star Oil | Anise Star Oil | 5.22% | 99.00% | 26.10% | 78.30% | 39.15% | 65.25% | 46.98% | 57.42% | 52.20% |
| Isoflavone | Genistein | 0.001% | 5.00% | 0.005% | 0.02% | 0.008% | 0.012% | 0.009% | 0.011% | 0.01 [symbol] |

Example 5 - Ingredient Family 5

| | | | | | | | | | | |
|---|---|---|---|---|---|---|---|---|---|---|
| Thyme Oil | Thyme Oil White | 2.05% | 61.50% | 10.25% | 30.75% | 15.38% | 25.63% | 18.45% | 22.55% | 20.5 [symbol] |
| Wintergreen Oil | Wintergreen Oil | 4.50% | 99.00% | 22.50% | 67.50% | 33.75% | 56.25% | 40.50% | 49.50% | 45.0 [symbol] |
| Vanillin | Vanillin | 0.11% | 5.00% | 0.55% | 1.65% | 0.83% | 1.38% | 0.99% | 1.21% | 1.10 [symbol] |
| Isopropyl myristate | Isopropyl myristate | 3.34% | 99.00% | 16.70% | 50.10% | 25.05% | 41.75% | 30.06% | 36.74% | 33.40% |

Example 6 - Ingredient Family 6

| | | | | | | | | | | |
|---|---|---|---|---|---|---|---|---|---|---|
| Limonene | D-Limonene | 5.63% | 99.00% | 28.15% | 84.45% | 42.23% | 70.38% | 50.67% | 61.93% | 56.3 [symbol] |
| Thyme Oil | Thyme Oil White | 1.24% | 37.14% | 6.19% | 18.57% | 9.29% | 15.48% | 11.14% | 13.62% | 12.3 [symbol] |
| Wintergreen Oil | Wintergreen Oil | 3.13% | 93.96% | 15.66% | 46.98% | 23.49% | 39.15% | 28.19% | 34.45% | 31.3 [symbol] |

Example 7 - Ingredient Family 7

| | | | | | | | | | | |
|---|---|---|---|---|---|---|---|---|---|---|
| Potassium Sorbate | Potassium Sorbate | 0.10% | 5.00% | 0.50% | 1.50% | 0.75% | 1.25% | 0.90% | 1.10% | 1.00% |
| Xanthan Gum | Xanthan Gum | 0.03% | 5.00% | 0.14% | 0.42% | 0.21% | 0.35% | 0.25% | 0.31% | 0.28% |
| Water | Water | 8.18% | 99.00% | 40.91% | 99.00% | 61.37% | 99.00% | 73.64% | 90.00% | 81.82% |
| Blend 74 | Blend 74 | 1.69% | 50.7% | 8.45% | 25.35% | 12.68% | 21.13% | 15.21% | 18.59% | 16.90% |

Example 8 - Ingredient Family 8

| | | | | | | | | | | |
|---|---|---|---|---|---|---|---|---|---|---|
| Isopropyl myristate | Isopropyl myristate | 4.84% | 99.00% | 24.18% | 72.53% | 36.26% | 60.44% | 43.52% | 53.19% | 48.35% |
| Geraniol | Geraniol Fine FCC | 1.50% | 44.94% | 7.49% | 22.47% | 11.24% | 18.73% | 13.48% | 16.48% | 14.98% |
| Blend 68 | Blend 68 | 3.67% | 99.00% | 18.34% | 55.01% | 27.50% | 45.84% | 33.00% | 40.34% | 36.67% |

Example 9 - Ingredient Family 9

| | | | | | | | | | | |
|---|---|---|---|---|---|---|---|---|---|---|
| Limonene | D-Limonene | 0.99% | 29.70% | 4.95% | 14.85% | 7.43% | 12.38% | 8.91% | 10.89% | 9.90% |
| Linalool | Linalool Coeur | 1.41% | 42.42% | 7.07% | 21.21% | 10.61% | 17.68% | 12.73% | 15.55% | 14.14% |
| Tetrahydrolinalool | Tetrahydrolinalool | 2.43% | 72.87% | 12.15% | 36.44% | 18.22% | 30.36% | 21.86% | 26.72% | 24.29% |
| Vanillin | Vanillin | 0.25% | 7.44% | 1.24% | 3.72% | 1.86% | 3.10% | 2.23% | 2.73% | 2.48% |
| Isopropyl myristate | Isopropyl myristate | 2.89% | 86.76% | 14.46% | 43.38% | 21.69% | 36.15% | 26.03% | 31.81% | 28.92% |
| Piperonal | Piperonal (aldehyde) | 1.00% | 29.91% | 4.99% | 14.96% | 7.48% | 12.46% | 8.97% | 10.97% | 9.97 [symbol] |
| Geraniol | Geraniol Fine FCC | 1.03% | 30.90% | 5.15% | 15.45% | 7.73% | 12.88% | 9.27% | 11.33% | 10.3 [symbol] |

Example 10 - Ingredient Family 10

| | | | | | | | | | | |
|---|---|---|---|---|---|---|---|---|---|---|
| Limonene | D-Limonene | 2.85% | 85.38% | 14.23% | 42.69% | 21.35% | 35.58% | 25.61% | 31.31% | 28.46 [symbol] |
| Thyme Oil | Thyme Oil White | 3.13% | 93.87% | 15.65% | 46.94% | 23.47% | 39.11% | 28.16% | 34.42% | 31.29 [symbol] |
| Blend 63 | Blend 63 | 4.03% | 99.00% | 20.13% | 60.38% | 30.19% | 50.31% | 36.23% | 44.28% | 40.25% |

Example 11 - Ingredient Family 11

| | | | | | | | | | | |
|---|---|---|---|---|---|---|---|---|---|---|
| Limonene | D-Limonene | 0.96% | 28.89% | 4.82% | 14.45% | 7.22% | 12.04% | 8.67% | 10.59% | 9.63 [symbol] |
| BSO | BSO | 2.67% | 79.98% | 13.33% | 39.99% | 20.00% | 33.33% | 23.99% | 29.33% | 26.66 [symbol] |
| Linalool | Linalool Coeur | 0.98% | 29.46% | 4.91% | 14.73% | 7.37% | 12.28% | 8.84% | 10.80% | 9.82 [symbol] |
| Tetrahydrolinalool | Tetrahydrolinalool | 1.18% | 35.43% | 5.91% | 17.72% | 8.86% | 14.76% | 10.63% | 12.99% | 11.81 [symbol] |
| Vanillin | Vanillin | 0.12% | 5.00% | 0.60% | 1.80% | 0.90% | 1.50% | 1.08% | 1.32% | 1.20% |
| Base Oil | Mineral Oil | 1.50% | 44.91% | 7.49% | 22.46% | 11.23% | 18.71% | 13.47% | 16.47% | 14.97% |
| | White USP | | | | | | | | | |
| Isopropyl myristate | Isopropyl myristate | 1.45% | 43.62% | 7.27% | 21.81% | 10.91% | 18.18% | 13.09% | 15.99% | 14.54% |
| Piperonal | Piperonal (aldehyde) | 0.49% | 14.55% | 2.43% | 7.28% | 3.64% | 6.06% | 4.37% | 5.34% | 4.85% |
| Geraniol | Geraniol Fine FCC | 0.65% | 19.53% | 3.26% | 9.77% | 4.88% | 8.14% | 5.86% | 7.16% | 6.51% |

Example 12 - Ingredient Family 12

| | | | | | | | | | | |
|---|---|---|---|---|---|---|---|---|---|---|
| Thyme Oil | Thyme Oil White | 4.19% | 99.00% | 20.93% | 62.79% | 31.40% | 52.33% | 37.67% | 46.05% | 41.86% |
| Isopropyl myristate | Isopropyl myristate | 3.83% | 99.00% | 19.17% | 57.51% | 28.76% | 47.93% | 34.51% | 42.17% | 38.34% |
| Geraniol | Geraniol Fine FCC | 1.98% | 59.40% | 9.90% | 29.70% | 14.85% | 24.75% | 17.82% | 21.78% | 19.80% |

Example 13 - Ingredient Family 13

| | | | | | | | | | | |
|---|---|---|---|---|---|---|---|---|---|---|
| Linalool | Linalool Coeur | 2.34% | 70.14% | 11.69% | 35.07% | 17.54% | 29.23% | 21.04% | 25.72% | 23.38% |
| Amyl Butyrate | Amyl Butyrate | 2.35% | 70.38% | 11.73% | 35.19% | 17.60% | 29.33% | 21.11% | 25.81% | 23.46% |
| Anise Star Oil | Anise Star Oil | 5.32% | 99.00% | 26.58% | 79.74% | 39.87% | 66.45% | 47.84% | 58.48% | 53.16% |

Example 14 - Ingredient Family 14

| | | | | | | | | | | |
|---|---|---|---|---|---|---|---|---|---|---|
| Linalool | Linalool Coeur | 3.74% | 99.00% | 18.72% | 56.16% | 28.08% | 46.80% | 33.70% | 41.18% | 37.44 [symbol] |
| Thymol | Thymol | 3.67% | 99.00% | 18.36% | 55.08% | 27.54% | 45.90% | 33.05% | 40.39% | 36.72 [symbol] |
| Pinene | Alpha-pinene, 98% | 0.47% | 13.98% | 2.33% | 6.99% | 3.50% | 5.83% | 4.19% | 5.13% | 4.66 [symbol] |
| Cymene | Para-Cymene | 0.19% | 5.61% | 0.94% | 2.81% | 1.40% | 2.34% | 1.68% | 2.06% | 1.87 [symbol] |
| Anethole | Trans-Anethole | 1.93% | 57.93% | 9.66% | 28.97% | 14.48% | 24.14% | 17.38% | 21.24% | 19.31 [symbol] |

Example 15 - Ingredient Family 15

| | | | | | | | | | | |
|---|---|---|---|---|---|---|---|---|---|---|
| Limonene | D-Limonene | 2.74% | 82.05% | 13.68% | 41.03% | 20.51% | 34.19% | 24.62% | 30.09% | 27.35% |
| Thyme Oil | Thyme Oil White | 3.01% | 90.24% | 15.04% | 45.12% | 22.56% | 37.60% | 27.07% | 33.09% | 30.08% |
| Lilac Flower Oil | Lilac Flower Oil | 4.26% | 99.00% | 21.30% | 63.90% | 31.95% | 53.25% | 38.34% | 46.86% | 42.57 [symbol] |

Example 16 - Ingredient Family 16

| | | | | | | | | | | |
|---|---|---|---|---|---|---|---|---|---|---|
| Thyme Oil | Thyme Oil White | 3.82% | 99.00% | 19.11% | 57.32% | 28.66% | 47.76% | 34.39% | 42.03% | 38.21 [symbol] |
| Wintergreen Oil | Wintergreen Oil | 2.48% | 74.37% | 12.40% | 37.19% | 18.59% | 30.99% | 22.31% | 27.27% | 24.79% |
| Isopropyl Myristate | Isopropyl Myristate | 3.59% | 99.00% | 17.95% | 53.84% | 26.92% | 44.86% | 32.30% | 39.48% | 35.89% |
| vanillin | Vanillin | 0.11% | 5.00% | 0.56% | 1.67% | 0.83% | 1.39% | 1.00% | 1.22% | 1.11% |

Example 17 - Ingredient Family 17

| | | | | | | | | | | |
|---|---|---|---|---|---|---|---|---|---|---|
| Wintergreen Oil | Wintergreen Oil | 2.48% | 74.46% | 12.41% | 37.23% | 18.62% | 31.03% | 22.34% | 27.30% | 24.82% |
| Isopropyl Myristate | Isopropyl Myristate | 3.59% | 99.00% | 17.97% | 53.91% | 26.96% | 44.93% | 32.35% | 39.53% | 35.94% |
| Thyme Oil | Thyme Oil White | 3.92% | 99.00% | 19.62% | 58.86% | 29.43% | 49.05% | 35.32% | 43.16% | 39.24% |

Example 18 - Ingredient Family 18

| | | | | | | | | | | |
|---|---|---|---|---|---|---|---|---|---|---|
| Thyme Oil | Thyme Oil White | 0.46% | 13.8% | 2.30% | 6.90% | 3.45% | 5.75% | 4.14% | 5.06% | 4.60% |
| Wintergreen Oil | Wintergreen Oil | 5.78% | 99.00% | 28.90% | 86.70% | 43.35% | 72.25% | 52.02% | 63.58% | 57.80% |
| Isopropyl Myristate | Isopropyl Myristate | 3.76% | 99.00% | 18.80% | 56.40% | 28.20% | 47.00% | 33.84% | 41.36% | 37.60% |

Example 19 - Ingredient Family 19

| | | | | | | | | | | |
|---|---|---|---|---|---|---|---|---|---|---|
| Thyme Oil | Thyme Oil White | 3.16% | 94.71% | 15.79% | 47.36% | 23.68% | 39.46% | 28.41% | 34.73% | 31.57% |
| Isopropyl myristate | Isopropyl myristate | 3.86% | 99.00% | 19.28% | 57.84% | 28.92% | 48.20% | 34.70% | 42.42% | 38.56% |
| Wintergreen Oil | Wintergreen Oil | 2.99% | 89.61% | 14.94% | 44.81% | 22.40% | 37.34% | 26.88% | 32.86% | 29.87 [symbol] |

Example 20 - Ingredient Family 20

| | | | | | | | | | | |
|---|---|---|---|---|---|---|---|---|---|---|
| Thyme Oil | Thyme Oil White | 2.06% | 61.80% | 10.30% | 30.90% | 15.45% | 25.75% | 18.54% | 22.66% | 20.60 [symbol] |
| Isopropyl myristate | Isopropyl myristate | 3.43% | 99.00% | 17.15% | 51.45% | 25.73% | 42.88% | 30.87% | 37.73% | 34.30 [symbol] |
| Geraniol | Geraniol Fine FCC | 4.51% | 99.00% | 22.55% | 67.65% | 33.83% | 56.38% | 40.59% | 49.61% | 45.10 [symbol] |

Example 21 - Ingredient Family 21

| | | | | | | | | | | |
|---|---|---|---|---|---|---|---|---|---|---|
| LFO | LFO | 5.01% | 99.00% | 25.07% | 75.20% | 37.60% | 62.66% | 45.12% | 55.14% | 50.13 |
| BSO (Black Seed Oil) | BSO | 4.99% | 99.00% | 24.94% | 74.81% | 37.40% | 62.34% | 44.88% | 54.86% | 49.87 |

Example 22 - Ingredient Family 22

| | | | | | | | | | | |
|---|---|---|---|---|---|---|---|---|---|---|
| LFO | LFO | 8.01% | 99.00% | 40.05% | 99.00% | 60.07% | 99.00% | 72.08% | 88.10% | 80.09% |
| BSO (Black Seed Oil) | BSO | 1.99% | 59.73% | 9.96% | 29.87% | 14.93% | 24.89% | 17.92% | 21.90% | 19.91% |

Example 23 - Ingredient Family 23

| | | | | | | | | | | |
|---|---|---|---|---|---|---|---|---|---|---|
| Geraniol | Geraniol Fine FCC | 2.90% | 57.00% | 14.95% | 45.00% | 22.42% | 37.38% | 26.90% | 32.90% | 29.90% |
| Isopropyl myristate | Isopropyl myristate | 3.85% | 73.15% | 19.25% | 57.75% | 28.87% | 48.13% | 34.65% | 42.35% | 38.50% |
| Thyme Oil | Thyme Oil White | 3.10% | 60.05% | 15.80% | 47.50% | 23.70% | 39.50% | 28.44% | 34.76 | 31.60% |

Example 24 - Ingredient Family 24

| | | | | | | | | | | |
|---|---|---|---|---|---|---|---|---|---|---|
| Isopropyl myristate | Isopropyl myristate | 3.40% | 65.15% | 17.15% | 51.45% | 25.70% | 42.90% | 30.85% | 37.75% | 34.29% |
| Wintergreen Oil | Wintergreen Oil | 4.50% | 85.71% | 22.56 | 67.70% | 33.85% | 56.40% | 40.50% | 50.00% | 45.11% |
| Blend 68 | Blend 68 | 2.00% | 40.00% | 10.30% | 30.89% | 15.40% | 25.75% | 18.50% | 22.65% | 20.59% |

Example 25 - Ingredient Family 25

| | | | | | | | | | | |
|---|---|---|---|---|---|---|---|---|---|---|
| Geraniol | Geraniol Fine FCC | 0.45% | 9.10% | 2.38% | 7.15% | 3.55% | 5.95% | 4.25% | 5.25% | 4.76% |
| Isopropyl myristate | Isopropyl myristate | 0.90% | 17.50% | 4.60% | 13.85% | 6.90% | 11.51% | 8.29% | 10.15% | 9.21% |
| Linalool | Linalool coeur | 0.65% | 12.50% | 3.25% | 9.80% | 4.90% | 8.20% | 5.85% | 7.20% | 6.53% |
| Piperonal | Piperonal (aldehyde) | 1.00% | 20.60% | 5.40% | 16.25% | 8.10% | 13.55% | 9.75% | 11.91% | 10.8 [symbol] |
| Tetrahydrolinalool | Tetrahydrolinalool | 0.85% | 17.10% | 4.45% | 13.50% | 6.70% | 11.21% | 8.05% | 9.875 | 8.97 [symbol] |
| Thyme Oil | Thyme Oil White | 5.00% | 95.00% | 24.95% | 75.00% | 37.45% | 62.45% | 44.90% | 54.95% | 49.93 [symbol] |
| Triethyl citrate | Triethyl citrate | 0.90% | 17.60% | 4.60% | 13.86% | 6.90% | 11.55% | 8.30% | 10.20% | 9.24 [symbol] |
| Vanillin | Vanillin | 0.05% | 5.00% | 0.27% | 0.81% | 0.40% | 0.68% | 0.49% | 0.59% | 0.54 [symbol] |

Example 26 - Ingredient Family 26

| | | | | | | | | | | |
|---|---|---|---|---|---|---|---|---|---|---|
| Limonene | D-Limonene | 0.85% | 16.60% | 4.35% | 13.10% | 6.50% | 10.90% | 7.85% | 9.60% | 8.7 [symbol] |
| Lilac Flower Oil | Lilac Flower Oil | 1.25% | 24.60% | 6.45% | 19.45% | 9.70% | 16.25% | 11.65% | 14.25% | 12.9 [symbol] |
| Lime Oil | Lime oil | 6.85% | 99.00% | 34.35% | 99.00% | 51.55% | 85.95% | 61.85% | 75.65% | 68.7 [symbol] |
| Thyme Oil | Thyme Oil White | 0.95% | 18.20% | 4.75% | 14.40% | 7.15% | 12.00% | 8.60% | 10.55% | 9.5 [symbol] |

Example 27 - Ingredient Family 27

| | | | | | | | | | | |
|---|---|---|---|---|---|---|---|---|---|---|
| Limonene | D-Limonene | 8.20% | 99.00% | 41.15% | 99.00% | 61.70% | 99.00% | 74.00% | 90.55% | 82.30% |
| Lilac Flower Oil | Lilac Flower Oil | 0.40% | 8.40% | 2.20 | 6.60% | 3.30% | 5.50% | 3.95% | 4.85% | 4.40% |
| Lime Oil | Lime oil | 1.00% | 19.00% | 5.00% | 15.00% | 7.50% | 12.50% | 9.00% | 11.00% | 10.00% |
| Thyme Oil | Thyme Oil White | 0.30% | 6.30% | 1.65% | 5.00% | 2.45% | 4.15% | 2.95% | 3.65% | 3.30% |

Example 28 - Ingredient Family 28

| | | | | | | | | | | |
|---|---|---|---|---|---|---|---|---|---|---|
| Benzyl alcohol | Benzyl alcohol | 5.40% | 99.00% | 27.00% | 81.05% | 40.51% | 67.51% | 48.60% | 59.45% | 54.01% |
| Limonene | D-Limonene | 1.30% | 25.00% | 6.50% | 19.65% | 9.80% | 16.40% | 11.75% | 14.40% | 13.09% |
| Thyme Oil | Thyme Oil White | 1.40% | 27.35% | 7.15% | 21.60% | 10.75% | 18.00% | 12.95% | 15.85% | 14.39% |
| Blend 63 | Blend 63 | 1.85% | 35.20% | 9.25% | 27.80% | 13.85% | 23.15% | 16.65% | 20.36% | 18.51% |

Example 29 - Ingredient Family 29

| | | | | | | | | | | |
|---|---|---|---|---|---|---|---|---|---|---|
| Benzyl alcohol | Benzyl alcohol | 5.40% | 99.00% | 27.00% | 81.05% | 40.51% | 67.51% | 48.60% | 59.45% | 54.01% |
| Limonene | D-Limonene | 1.30% | 25.00% | 6.50% | 19.65% | 9.80% | 16.40% | 11.75% | 14.40% | 13.09% |
| Geraniol | Geraniol Fine FCC | 0.15% | 3.35% | 0.85% | 2.65% | 1.30% | 2.20% | 1.55% | 1.95% | 1.76% |
| Isopropyl myristate | Isopropyl myristate | 0.30% | 6.50% | 1.70% | 5.10% | 2.55% | 4.25% | 3.05% | 3.75% | 3.40% |
| Linalool | Linalool Coeur | 0.20% | 4.60% | 1.20% | 3.62% | 1.80% | 3.05% | 2.15% | 2.65% | 2.41 [symbol] |
| Piperonal | Piperonal (aldehyde) | 0.40% | 7.60% | 2.00% | 6.00% | 3.00% | 5.00% | 3.60% | 4.40% | 4.00 [symbol] |
| Tetrahydrolinalool | Tetrahydrolinalool | 0.33% | 6.35% | 1.65% | 5.00% | 2.49% | 4.15% | 2.95% | 3.65% | 3.32 [symbol] |
| Thyme Oil | Thyme Oil White | 1.40% | 27.35% | 7.15% | 21.60% | 10.75% | 18.00% | 12.95% | 15.85% | 14.39 [symbol] |
| Triethyl Citrate | Triethyl Citrate | 0.34% | 6.48% | 1.70% | 5.12% | 2.55% | 4.30% | 3.05% | 3.75% | 3.41 [symbol] |
| Vanillin | Vanillin | 0.02% | 5.00% | 0.10% | 0.30% | 0.15% | 0.25% | 0.18% | 0.22% | 0.20 [symbol] |

Example 30 - Ingredient Family 30

| | | | | | | | | | | |
|---|---|---|---|---|---|---|---|---|---|---|
| Black Seed Oil | Black Seed Oil | 0.26% | 50.00% | 13.10% | 39.30% | 19.65% | 32.75% | 23.55% | 28.85% | 26.2 [symbol] |
| Limonene | D-Limonene | 0.85% | 16.75% | 4.40% | 13.20% | 6.60% | 11.00% | 7.90% | 9.70% | 8.80 [symbol] |
| Geraniol | Geraniol Fine FCC | 0.40% | 8.20% | 2.15% | 6.45% | 3.20% | 5.40% | 3.85% | 5.05% | 4.30 [symbol] |
| Isopropyl myristate | Isopropyl myristate | 0.95% | 18.05% | 4.75% | 14.25% | 7.10% | 11.90% | 8.55% | 10.45% | 9.50 [symbol] |
| Linalool | Linalool Coeur | 0.64% | 12.20% | 3.20% | 9.60% | 4.80% | 8.00% | 5.75% | 7.05% | 6.40 [symbol] |
| Methyl Salicyclate | Methyl Salicyclate | 3.30% | 62.70% | 16.50% | 49.50% | 24.75% | 41.25% | 29.70% | 36.3% | 33.0 [symbol] |
| Piperonal | Piperonal (aldehyde) | 0.30% | 6.10% | 1.60% | 4.80% | 2.40% | 4.00% | 2.85% | 3.55% | 3.20% |
| Tetrahydrolinalool | Tetrahydrolinalool | 0.75% | 14.85% | 3.90% | 11.70% | 5.85% | 9.75% | 7.00% | 8.60% | 7.80% |
| Vanillin | Vanillin | 0.05% | 5.00% | 0.40% | 1.20% | 0.60% | 1.00% | 0.72% | 0.88% | 0.80% |

[symbol]

indicates data missing or illegible when filed

In addition, embodiments are specifically contemplated in which any of the ingredients of the above lists are combined, with the proviso that the ingredients do not comprise any of the combinations of specific ingredients shown in a row of Table 3. Furthermore, embodiments are specifically contemplated in which any of the ingredients listed in Table 1 are combined, with the proviso that the ingredients do not comprise any of the combinations of specific ingredients shown in a row of Table 3. Moreover, embodiments are specifically contemplated in which any of the ingredients listed in any one of Blends 1-126 of Table 1 are combined, with the proviso that the ingredients do not comprise any of the combinations of specific ingredients shown in a row of Table 3. Finally, embodiments are specifically contemplated in which any of the ingredients listed in Table 2 are combined, with the proviso that the ingredients do not comprise any of the combinations of specific ingredients shown in a row of Table 3. Thus, for example, an embodiment is contemplated in which any of these ingredients are combined, with the proviso that the ingredients do not comprise α-terpineol or pyrethrum.

TABLE 3
Specific Ingredient Combinations Excluded From Embodiments of the Invention

α-terpineol and pyrethrum
Benzyl alcohol and eugenol
Benzyl alcohol and cis-jasmone
Benzyl alcohol and tetrahydrofurfuryl alcohol
Eugenol and pyrethrum
Thyme oil and pyrethrum
Thymol and carbaryl
Thymol and chrysanthemate ester
Thymol, chrysanthemyl alcohol, and chrysanthemic acid
Thymol, eugenol, trans-anethole, α-terpineol, citronellal, and propargite
Thymol, eugenol, trans-anethole, α-terpineol, citronellal, and pyrethrum
Thymol, eugenol, trans-anethole, α-terpineol, citronellal, and tebufenozide
Thymol and cis-jasmone
Thymol and deltamethrin
Thymol and lavandustin A
Thymol and PD 98059
Thymol and permethrin
Thymol and pyrethrum
Benzyl alcohol and pyrethrum
Benzyl alcohol and chrysanthemate ester
Benzyl alcohol, chrysanthemyl alcohol, and chrysanthemic acid
Benzyl alcohol and trans-anethole
Benzyl alcohol, tetrahydrofurfuryl alcohol, PD 98059, trans-anethole, and chrysanthemate ester
Benzyl alcohol, tetrahydrofurfuryl alcohol, PD 98059, trans-anethole, and pyrethrum
Eugenol, phenylethyl propionate, and menthyl salicylate
Phenylethyl alcohol, α-terpineol, and benzyl alcohol
Phenylethyl alcohol, α-terpineol, benzyl alcohol, and phenylethyl propionate
Phenylethyl alcohol, α-terpineol, benzyl alcohol, phenylethyl propionate, and eugenol
Tamoxifen and forskolin
Thymol and benzyl alcohol
Thymol, eugenol, trans-anethole, α-terpineol, citronellal, and chrysanthemate ester
Thymol, eugenol, trans-anethole, α-terpineol, citronellal, and chrysanthemic acid
Thymol, eugenol, trans-anethole, α-terpineol, citronellal, and chrysanthemyl alcohol
Thymol, eugenol, trans-anethole, α-terpineol, citronellal, and cis-jasmone

In some embodiments, each compound can make up between about 1% to about 99%, by weight (wt/wt %) or by volume (vol/vol %), of the composition. For example, one composition of the present invention comprises about 2% alpha-Pinene and about 98% D-limonene. As used herein, percent amounts, by weight or by volume, of compounds are to be understood as referring to relative amounts of the compounds. As such, for example, a composition including 7% linalool, 35% thymol, 4% alpha-pinene, 30% para-cymene, and 24% soy bean oil (vol/vol %) can be said to include a ratio of 7 to 35 to 4 to 30 to 24 linalool, thymol, alpha-pinene, para-cymene, and soy bean oil, respectively (by volume). As such, if one compound is removed from the composition, or additional compounds or other ingredients are added to the composition, it is contemplated that the remaining compounds can be provided in the same relative amounts. For example, if soy bean oil were removed from the exemplary composition, the resulting composition would include 7 to 35 to 4 to 40 linalool, thymol, alpha-pinene, and para-cymene, respectively (by volume). This resulting composition would include 9.21% linalool, 46.05% thymol, 5.26% alpha-pinene, and 39.48% para-cymene (vol/vol %). For another example, if safflower oil were added to the original composition to yield a final composition containing 40% (vol/vol) safflower oil, then the resulting composition would include 4.2% linalool, 21% thymol, 2.4% alpha-pinene, 18% para-cymene, 14.4% soy bean oil, and 40% safflower oil (vol/vol %). One having ordinary skill in the art would understand that volume percentages are easily converted to weight percentages based the known or measured specific gravity of the substance.

In certain embodiments, it can be desirable to include a naturally-occurring version or a synthetic version of a compound. In certain exemplary compositions, it can be desirable to include a compound that is designated as meeting Food Chemical Codex (FCC), for example, Geraniol Fine FCC or Tetrahydrolinalool FCC, which compounds can be obtained, for example, from Millennium Chemicals, Inc.

In certain embodiments, it can be desirable to combine an insect control blend as described herein with a synthetic insecticide such as pyrethroid compound, a nitroguanidine compound or a chloronicotinyl compound. For example, in certain embodiments it can be desirable to combine a blend with deltamethrin, clothianidin or imidacloprid, or a combination thereof. Deltamethrin is available for example from AgrEvo Environmental Health, Inc., of Montvale, N.J. Clothianidin and imidacloprid are available from Bayer CropScience LP of Research Triangle Park, N.C.

In embodiments of the invention that include at least one blend of compounds of a plant origin, the compounds of plant origin can be tested for their precise chemical composition using, for example, High-Pressure Liquid Chromatography (HPLC), Mass Spectrometry (MS), gas chromatography, or the like.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” means within an acceptable error range for the particular value.

The term “substantially,” as used herein, means at least about 80%, preferably at least about 90%, more preferably at least about 99%, for example at least about 99.9%. In some embodiments, the term “substantially” can mean completely, or about 100%.

The term “substantially unaffected,” as used herein, means exhibiting no adverse effects at a dosage level at which a reference organism, such as a target pest, does exhibit such effects.

The term “complementary effects” includes synergistic effects.

Embodiments of the invention can include at least one oil, such as, for example, “Superior oil,” highly-refined oils, and the like.

In the case of an animal, human or non-human, the host can also be treated directly by using a formulation of a composition that is delivered orally. For example, a composition can be enclosed within a liquid capsule and ingested.

An area can be treated with a composition of the present invention, for example, by using a spray formulation, such as an aerosol or a pump spray, or a burning formulation, such as a candle or a piece of incense containing the composition. Of course, various treatment methods can be used without departing from the spirit and scope of the present invention. For example, compositions can be comprised in household products such as, for example, air fresheners (including heated air fresheners in which insect repellent substances are released upon heating, e.g., electrically, or by burning); hard surface cleaners; laundry products (e.g., laundry detergent-containing compositions, conditioners), and the like.

In certain embodiments, the insect control composition can selectively control insects of a target species while delaying or preventing the onset of resistance to the composition. The development of resistance in insects to acutely toxic insecticides generally occurs by selection of individuals in a population that can survive the insecticide. It is pre-adaptive and not a mutational effect. This implies that it is an inherited trait. Most commercial insecticides are designed to be poor mutagens and their use results in an intense chemical selection (high dose, high toxicity) which is not conducive to genetic alterations, but allows survival of pre-adapted (i.e. resistant) individuals. Species can become resistant either behaviorally (acting to avoid exposure to a lethal dose) or physiologically (by developing ways to survive a normally lethal dose).

There are a number of different mechanisms by which physiological resistance can occur. Mechanisms can include reduced penetration (of the insecticide through the cuticle), target site resistance (i.e. the target site is no longer affected by the insecticide), increased metabolic detoxification (so that the pesticide is detoxified as a result of enhanced levels or modified activities of esterases, oxidases, or glutathione S-transferases (GST) before it reaches the target site), sequestration of the pesticide (i.e. stored in the body where it is not harmful) and possible increased excretion of the ingested pesticide. In a population of insects, it is often a combination of factors which greatly results in the overall expression of resistance.

Embodiments of the invention can mitigate these resistance mechanisms in a number of ways. In certain embodiments of the invention, cuticle penetration is not necessary for effective insect control, as the insect control composition can reach the target via olfactory receptors.

Additionally, compositions of the invention and components thereof can cause an immediate cellular effect on target species, thus mitigating the effects of elevated excretion or sequestration.

In certain embodiments, compositions of certain embodiments of the inventions are resistant to enzymatic degradation, because components of the compositions can act as receptor ligand analogs. In other embodiments, the compositions of the invention can act on multiple target sites, thus reducing the selective pressure upon any single target site, and thus minimizing the chance of resistance development in the target pest.

For example, the multiple targets acted upon by low-resistance pest control compositions of embodiments of the invention simultaneously act on two or more receptors or other molecular targets. These include, for example, tyramine receptor, octopamine receptor, olfactory receptor Or83b, olfactory receptor 43a, serotonin receptor, Or22a, Or22b, Gr5a, Gr21a, Gr61a, β-arrestin receptor, GRK2 receptor, tyramine β-hydroxylase receptor, DAF-2, DAF-12, E78, E75, DHR3, EcR, DHR96, USP, DHR78, HNF4, SVP, TLL, DSF, DHR51, DHR83; ERR, DHR38; FTZ-F1, DHR39; DIIR4, PPAR, RAR, TR, REV-ERB, ROR, FXR, LXR, VDR, SXR, CAR; RXR, TR2/TR4, HNF4, COUP-TF, TLX, PNR; ERR, ER, MR/PR/AR/GR, NURR1/NGFIB, LRH/SFl, and GCNF.

In other embodiments, the targets may include invertebrate or parasite nuclear receptor proteins having as their native ligand naturally occurring hormones such as la, 25(OH)2-vitamin D3,17p-oestradiol, testosterone, progesterone, cortisol, aldosterone, all-trans retinoic acid, 3,5,3′-L-triiodothyronine, cc-ecdysone, or brassinolide, among others.

Other targets that are acted upon by embodiments of the present invention include: sialidase, serine-rich E. histolytica protein (SREHP), amebic galactose-specific lectin, galactose/N-acetyl-D-galactosamine-inhibitable lectin (Gal-lectin), KERP1, pyruvate phosphate dikinase, glyceraldehyde-3-phosphate dehydrogenase, and 140 kDaFN-binding molecule (EhFNR) (all as found, for example, in Entamoeba histolytica and in other organisms); aurora kinase, α 14-Giardin (annexin El), dynamin-related protein (G1DRP), Nitroreductase (G1NR1), and UDP-N-acetylglucosamine 4′-epimerase (all as found, for example, in Giardia lamblia and in other organisms); CM250 (as found, for example, in Cryptosporidium muris and in other organisms); thrombospondin-related protein CpMICl (CpTSP8), p30, and Cpa135 (all as found, for example, in Cryptosporidium parvum and in other organisms); TcRBP19, gp82 defined by monoclonal antibody 3F6, TcPINl, and metacaspases TcMCA3 and TcMCA5 (all as found, for example, in Trypanosomatidae cruzi and in other organisms); OP-Tb (as found, for example, in Leishmania brucei and in other organisms), major surface protease (MSP), UDP-galactopyranose mutase (GLF), and surface-metalloprotease (leishmanolysin) (as found, for example, in Leishmania spp.); rhoptry proteins (ROPs), MIC2, acyl carrier protein (ACP) (as found, for example, in. Toxoplasma gondii and in other organisms); thromobospondin-related sporozoite protein (TRSP), circumsporozoite protein (CSP), and duffy-binding-like erythrocyte-binding proteins (DBL-EBP) (as found, for example, in Plasmodium spp.); thrombospondin related adhesive proteins (TRAPS) (as found, for example, in Babesia); cysteine proteases (CPs) and AP65 (as found, for example, in Trichomonas vaginalis and in other organisms); Step 20 group Serine/threonine kinases (as found, for example, in Schistosoma spp.); Taenia adhesion family (TAF) (as found, for example, in Taenia spp.); flottillin-1 (as found, for example, in Eimeria spp.); excretory-secretory products (ESP) (as found, for example, in Fasciola spp.); extracellular avirulence proteins (avrs) (as found, for example, in Cladosporium spp.); pH-responsive PacC/RimlOl transcription regulators, ClaSSDl, and STE12-like genes (as found, for example, in Colletotrichum spp.); NcAMAl (as found, for example, in Neospora spp.); nucleoside triphosphate hydrolase (NTPase) (SnNTP1) (as found, for example, in Sarcocystis spp.); fer1, fer2, and bizl (as found, for example, in Ustilago maydis); snodprotl family (e.g., MSP1) and ABC transporters (ABC1) (as found, for example, in Magnaporthe grisea); secreted lipase (FGL1) and targets involved in deoxynivalenol biosynthesis (TRI14, TRIM) (as found, for example, in Fusarium spp.); and extracellular hydrolases (e.g. endopolygalacturonase P2c), gliP, and Asp-hemolysin (as found, for example, in Aspergillus spp.). Further information as to these targets may be found in International Patent Application No. PCT/US08/088,342, which is incorporated herein by reference in its entirety.

Other targets that are acted upon by embodiments of the present invention include the receptors and other molecular targets shown in Table 4 below. In the Table, a, b and g correspond to the Greek letters α,β and gamma, respectively.

TABLE 4

| Sub- | | | |
|---|---|---|---|
| families | | | |
| and | | | Accession |
| Group | Genes | Trivial Names | numbers |
| 1A | NR1A1 | thyroid hormone receptor, TRa, c- | M24748 |
| | | erbA-1, THRA | |
| | NR1A2 | thyroid hormone receptor, TRb, c- | X04707 |
| | | erbA-2, THRB | |
| IB | NR1B1 | retinoic acid receptor, RARa | X06538 |
| | NR1B2 | retinoic acid receptor, RARb, HAP | Y00291 |
| | NR1B3 | retinoic acid receptor, RARg, RARD | M57707 |
| | NR1B4 | retinoic acid receptor, RAR | AF378827 |
| 1C | NR1C1 | peroxisomeproliferator-activated | L02932 |
| | | receptor, PPARa | |
| | NR1C2 | peroxisomeproliferator-activated | L07592 |
| | | receptor, PPAR6, NUC1, PPARd, | |
| | | FAAR | |
| | NR1C3 | peroxisomeproliferator-activated | L40904 |
| | | receptor, PPARg | |
| ID | NR1D1 | reverse erbA, REVERBa, EAR1, | M24898 |
| | | EAR1A | |
| | NR1D2 | reverse erbA, REVERBb, EAR 1b, | L31785 |
| | | BD73, RVR, HZF2 | |
| | NR1D3 | reverse erbA, E75 | X51548 |
| IE | NR1E1 | E78, DR-78 | U01087 |
| IF | NR1F1 | RAR-related orphan receptor, RORa, | U04897 |
| | | RZRa | |
| | NR1F2; | RAR-related orphan receptor, RORb, | Y08639 |
| | | RZRb | |
| | NR1F3 | RAR-related orphan receptor, RORg, | U16997 |
| | | TOR | |
| | NR1F4 | HR3, DHR3, MHR3, GHR3 | M90806 |
| | | CNR3, CHR3 | U13075 |
| 1G | NR1G1 | CNR 14 | U13074 |
| 1H | NR1H1 | ECR | M74078 |
| | NR1H2 | Liver X receptor, UR, OR-1, NER1, | U07132 |
| | | RIP15, LXRb | |
| | NR1H3 | Liver X receptor, RLD1, LXR, LXRa | U22662 |
| | NR1H4 | Farnesoid X receptor, FXR, RIP14, | U09416 |
| | | HRR1 | |
| | NR1H5 | Farnesoid X receptor, FXRB | AY094586 |
| 11 | NR1I1 | Vitamin D receptor, VDR | J03258 |
| | NR1I2 | Pregnane X receptor, ONR1, PXR, | X75163 |
| | | SXR, BXR | |
| | NR1I3 | Constitutive androstane | Z30425 |
| | | receptor, MB67, CAR1, CARa | |
| | NR1I4 | CAR2, CARb | AF009327 |
| U | NR1J1 | DHR96 | U36792 |
| IK | NR1K1 | NHR1 | U19360 |
| 2A | NR2A1 | Human nuclear factor 4, HNF4 | X76930 |
| | NR2A2 | Human nuclear factor 4, HNF4G | Z49826 |
| | NR2A3 | HNF4B | Z49827 |
| | NR2A4 | DHNF4, HNF4D | U70874 |
| 2B | NR2B1 | Retinoid X receptor, RXRA | X52773 |
| | NR2B2 | Retinoid X receptor, RXRB, | M84820 |
| | | H-2RIIBP, RCoR-1 | |
| | NR2B3 | Retinoid X receptor, RXRG | X66225 |
| | NR2B4 | USP, Ultraspiracle, 2C1, CF1, | X52591 |
| | | RXR1, RXR2 | |
| 2C | NR2C1 | Testis receptor, TR2, TR2-11 | M29960 |
| | NR2C2 | Testis receptor, TR4, TAK1 | L27586 |
| | NR2C3 | TR2-4 | AF378828 |
| 2D | NR2D1 | DHR78 | U36791 |
| 2E | NR2E1 | TLL, TLX, XTLL | S72373 |
| | NR2E2 | TLL, Tailless | M34639 |
| | NR2E3 | Photoreceptor-specific | AF121129 |
| | | nuclear receptor, PNR | |
| | NR2E4 | dissatisfaction | 096680 |
| | NR2E5 | fax-1 | Q9U4I0 |
| 2F | NR2F1 | Chicken ovalbumin upstream | XI2795 |
| | | promoter-transcription | |
| | | factor, COUP-TFI, COUPTFA, | |
| | | EAR3, SVP44 | |
| | NR2F2 | Chicken ovalbumin upstream | M64497 |
| | | promoter-transcription | |
| | | factor, COUP-TFII, COUPTFB, | |
| | | ARP1, SVP40 | |
| | NR2F3 | SVP, COUP-TF | M28863 |
| | NR2F4 | COUP-TFIII, COUPTFG | X63092 |
| | NR2F5 | SVP46 | X70300 |
| | NR2F6 | ErbA2-related gene 2, EAR2 | XI2794 |
| | NR2F7 | AmNR7 | AF323687 |
| 2G | NR2G1 | HNF, RXR | AJ517420 |
| 2H | NR2H1 | AmNR4, AmNR8 | AF323683 |
| 3A | KR3A1 | ERa | X03635 |
| | NR3A2 | ERb | U57439 |
| 3B | NR3B1 | ERR1, ERRa | X514I6 |
| | NR3B2 | ERR2, ERRb | X514I7 |
| | NR3B3 | ERR3, ERRg | AF094318 |
| | NR3B4 | Drosophila ERR | AE003556 |
| 3C | NR3C1 | GR | X03225 |
| | NR3C2 | MR | M16801 |
| | NR3C3 | PR | M15716 |
| | NR3C4 | AR | M20132 |
| 4A | NR4A1 | NGFIB, TR3, N10, NUR77, NAK1 | LI3740 |
| | NR4A2 | NURR1, NOT, RNR1, HZF-3, | X75918 |
| | | TINOR | |
| | NR4A3 | NOR 1, MINOR | D38530 |
| | NR4A4 | DHR38, NGFIB | U36762 |
| | | CNR8, C48D5 | U13076 |
| 5A | NR5A1 | SF1, ELP, FTZ-F1, AD4BP | D88155 |
| | NR5A2 | LRH1, xFFlrA, xFFlrB, FFLR, | U93553 |
| | | PHR, FTF | |
| | NR5A3 | FTZ-F1 | M63711 |
| | NR5A4 | 4FFlb | Q9IAI9 |
| 5B | NR5B1 | DHR39, FTZF1B | L06423 |
| 6A | NR6A1 | GCNF1, RTR | U14666 |
| | NR6A2 | HR4, THR4, GRF | AL035245 |
| 0A | NROA1 | KNI, Knirps | X13331 |
| | NROA2 | KNRL, Knirps related | X14153 |
| | NROA3 | EGON, Embryonic gonad, EAGLE | X16631 |
| | NROA4 | ODR7 | U16708 |
| | NR0A5 | Trithorax | M31617 |
| 0B | NR0B1 | DAX1, AHCH | S74720 |
| | NR0B2 | SHP | L76571 |

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

An insect control composition was prepared according to the following:

| Ingredient | CAS | W/W |
|---|---|---|
| Thyme Oil White | 8007-46-3 | 20.6 |
| Isopropyl myristate | 110-27-0 | 34.3 |
| Wintergreen Oil | | 45.1 |

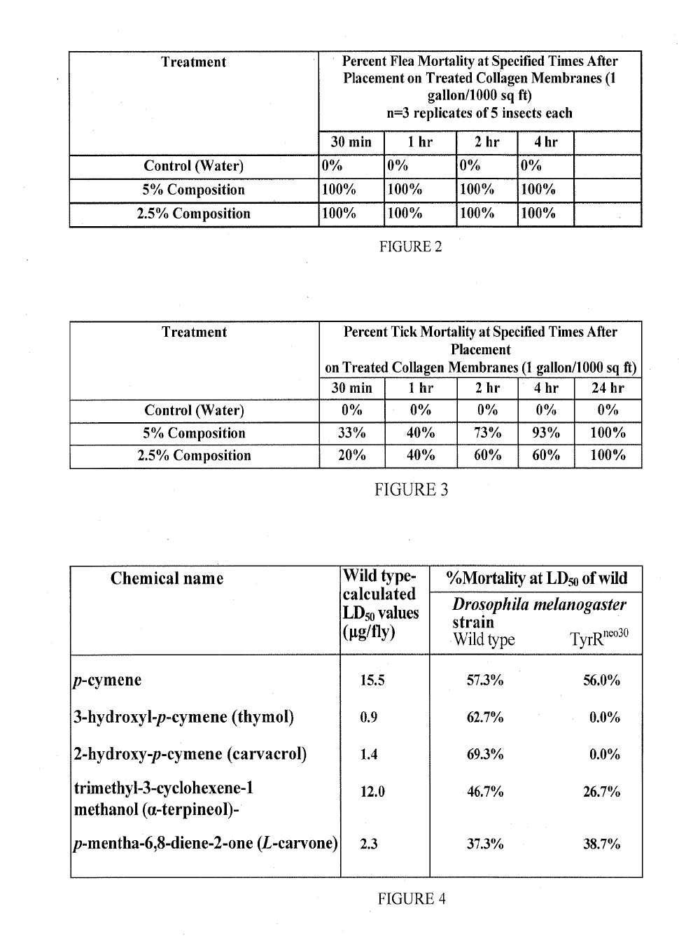

The composition was tested for insect control potential against fleas by adding the composition at various concentrations to collagen membranes (1 gallon per 1000 sq. ft.), and then placing fleas (n=3, replicates of 5 fleas each) upon the membranes. Flea mortality was measured at the time points indicated in FIG. 2.

Next, the composition was tested for insect control potential against ticks by adding the composition at various concentrations to collagen membranes (1 gallon per 1000 sq. ft.), and then placing fleas (n=3, replicates of 5 ticks each) upon the membranes. Tick mortality was measured at the time points indicated in FIG. 3.

The data reflects the selectivity of the test composition, as the percent mortality for fleas was 500% higher than the percent mortality for ticks at the 30 minute time point.

### Example 2

LD₅₀ values of tested chemicals (determined against wild type Drosophila) were topically applied against wild type and tyramine receptor mutant (TyrRneo30) strains. Mortality was determined 24 h after treatment. Data are the average of three replicates, 5 flies per replicate. This experiment was repeated five times.

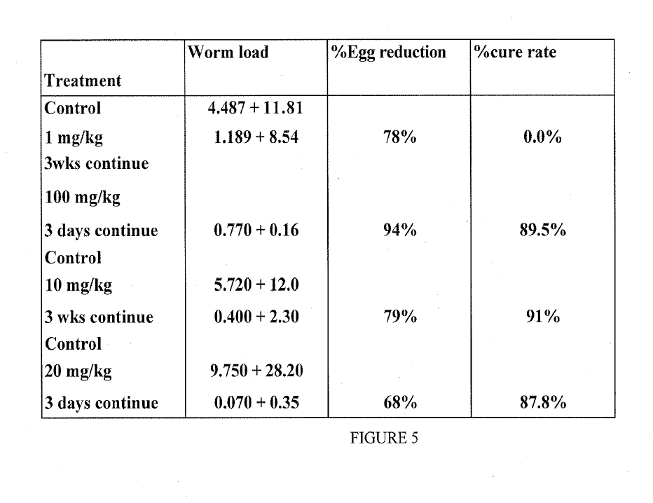

The data (FIG. 4) shows the selectivity of the thymol and carvacrol test chemicals, as the chemicals did not affect Drosophila expressing a mutant form of the tyramine receptor.

### Example 3

A parasite-control test composition was prepared according to Table 1.

Mice were treated with a test composition at various dose levels for various time periods (see FIG. 5). Briefly, test composition was administered in various amounts (1, 10, or 100 mg/kg of body weight) to mice for a period of either three days or three weeks, followed by infection of each animal with 200 viable eggs of H. nana. During the 2 week incubation period following infection, the animals continued to be treated with the blend compositions at the various test amounts. During the third week post-infection, the stool of the treated and infected mice was examined, and the mice were then sacrificed at the end of the third week to ascertain cure rate. A background infection number was established by infecting untreated animals with 200 viable eggs of H. nana.

The resulting data indicated that despite the compositions' effectiveness against the H. nana parasite, there were no significant differences between treated and untreated animals with regard to blood, fecal consistency, changes in water intake, changes in food intake, or changes in body weight. Further, no internal bleeding was found in any of infected/treated animals, nor were there any significant differences in liver or kidney function between treated and untreated animals. This data demonstrates the composition's safety toward vertebrates while also showing the efficacy of the composition against parasites.

### Example 4

When the food additive amendments to the Food, Drug, and Cosmetic Act (FDCA) were enacted in 1958, certain food ingredients with a long history of use were exempted from the pre-market evaluation and approval process required for food additives. Such compounds were labeled as “generally recognized as safe” (GRAS) under the conditions of their intended use. However, any food ingredient can be considered GRAS, as long as it is generally recognized among scientific experts (qualified by scientific training and experience) to be safe under the conditions of its intended use.

The FDA officially lists compounds it considers GRAS in 21 CFR Parts 182, 184, and 186. Over 680 substances are given. However, the list does not include every GRAS substance. The FDA realizes that it is impractical to list all such items, and emphasizes this is just a fraction of the compounds that can be considered GRAS. For example, in addition to these 680+ compounds, The Flavor and Extract Manufacturers' Association (“FEMA”) has independently affirmed over 2,000 compounds as GRAS. The FDA accepts the FEMA GRAS review process as consistent with the criteria set forth in the FICA and has, essentially, adopted the FEMA list as a de facto official FDA-approved list of GRAS flavoring agents. The ingredients listed in 21 CFR Parts 182, 184, and 186, and those listed in the FEMA GRAS lists 3 through 24 are contemplated for use in embodiments of the present invention, and those documents are incorporated by reference herein.

For example, one composition of the present invention includes about 1% thymol, about 50% geraniol and about 49% mineral oil. Additionally, it is contemplated that these compositions may be made up of generally regarded as safe (GRAS) compounds, for example: thyme oil, geraniol, lemon grass oil, lilac flower oil, black seed oil, lime oil, eugenol, castor oil, mineral oil, and safflower oil.

### Example 5

Cabbage leaves (Brassica oleracea) are treated by being dipped into a test composition selected from those set forth in Tables 1 and 2 at a desired concentration and have mustard beetle larvae (Phaedon cochleariae) placed on them while the leaves are still moist.

After the desired period of time, the percent mortality is determined for the insects, and any phytotoxic effects are quantified as well to determine the effect of the test compositions on non-target species. The data indicate that the test composition has a strongly pesticidal effect on the mustard beetle larvae, while having little or no effect on the cabbage leaves.

### Example 6

Soya bean shoots (glycine max) are treated by being dipped into a test composition of the desired concentration and are populated with Heliothis armigera caterpillars while the leaves are still moist.

After the desired period of time, the percent mortality is determined for the insects, and any phytotoxic effects are quantified as well to determine the effect of the test compositions on non-target species. The data indicate that the test composition has a strongly pesticidal effect on the caterpillars, while having little or no effect on the bean shoots.

### Example 7

Test compositions are evaluated for activity against various insects in a surface-treated diet test. In this test one mL of molten (65 70° C.) wheat germ-based artificial diet is pipetted into each well of a four by six (24 well) multi-well plate (ID# 430345-15.5 mm diameter×17.6 mm deep; Corning Costar Corp., One Alewife Center, Cambridge, Mass. 02140). The diet is allowed to cool to ambient temperature before treatment with the test composition.

For a determination of insecticidal activity, test compositions according to Table I are prepared for testing using a Packard 204DT Multiprobe® Robotic System (Packard Instrument Company, 800 Research Parkway, Meriden, Conn. 06450), in which the robot first dilutes a standard solution of test composition with a 1:1 water/acetone solution (V/V) in a ratio of 1:7 stock solution to water/acetone. The robot subsequently pipettes 40 uL of the solution onto the surface of the diet in each of three wells in the 24 multi-well plate. Once treated, the contents of the multi-well plate are allowed to dry, leaving 0.25 mmol of test composition on the surface of the diet, or a concentration of 0.25 millimolar. Appropriate untreated controls containing only DMSO on the diet surface are also included in this test.

For evaluations of the insecticidal activity of a test composition at varying rates of application, the test is established as described above using sub-multiples of the standard 50 millimolar DMSO solution of candidate insecticide. For example, the standard 50 millimolar solution is diluted by the robot with DMSO to give 5, 0.5, 0.05, 0.005, 0.0005 millimolar, or more dilute solutions of the test composition. In these evaluations there are six replicates of each rate of application placed on the surface of the diet in the 24 multi-well plate, for a total of four rates of application of test composition in each plate.

In each well of the test plate is placed one test insect. Several species are used. After the insects are placed in each well, the plate is sealed with clear polyfilm adhesive tape. The tape over each well is perforated to ensure an adequate air supply. The plates are then held in a growth chamber at 25° C. and 60% relative humidity for five days (light 14 hours/day).

After the five-day exposure period, insecticidal activity for each rate of application of test composition is assessed as percent inhibition of insect weight relative to the weight of insects from untreated controls, and percent mortality when compared to the total number of insects infested. These results allow comparison of test composition effectiveness between various insect species, as well as providing data that can be used to design compositions that can selectively affect the target insect species while minimally harming non-target species.

### Example 8

Bred green peach aphids (Myzus persicae) having resistance to organophosphate and carbamate chemicals are inoculated on eggplant (black elongate variety) seedlings, about 20 cm tall, grown in 15 cm unglazed pots, at a rate of about 200 per seedling. One day post-inoculation, a water dilution of a test composition in a predetermined concentration is sprayed in sufficient amounts by means of a spray gun. After the spraying, the pots are left to stand in a greenhouse kept at 28° C. Twenty-four hours after the spraying, the percent mortality is calculated. The above test is carried out through two replicates. The resulting data shows that the test compositions' efficacy is unaffected by insect resistance to organophosphate and carbamate chemicals, demonstrating that the mechanism of action employed by the test compositions differs from that employed by organophosphate and carbamate chemicals. Because of the difference in mechanisms of action, the test compositions are an effective component of a pest control regime designed to minimize resistance development in the target pest. Further, because the typical modes of resistance-development are less likely to result in resistance to the compositions of the invention, these compositions can delay, minimize, and prevent resistance development in the target pest.

### Example 9

Test compositions are applied to crops using a CO₂-pressurized backpack sprayer. The application methods are designed to simulate commercial ground application of crop protection products normally carried out with spray tractors equipped with a spray tank, a pressure pump and a spray rig with nozzles.

Test compositions are applied to cotton at the 2-3 true leaf growth stage (seedling plants) with the sprayer calibrated to deliver the appropriate amount of the test composition through two hollow cone nozzles. One plot (A) is left untreated as a control. Each test plot consists of a 65-ft long row of crop and each treatment is replicated four times in a randomized complete block design. Thrips are counted 1-9 days after treatment by collecting 10 cotton plants from each test plot at random. The collected plants are washed with a water and detergent plus bleach solution in the laboratory. The water is then poured onto a coffee filter and from there thrips are rinsed onto a filter paper to be counted under a scope. Phytotoxicity data is also collected at this time. The data indicate that the test composition has a strongly pesticidal effect on the thrips, while having little or no effect on the cotton plants.

### Example 10

Heliothis zea (cotton bollworm), Heliothis virescens (tobacco budworm) and pyrethroid-resistant Heliothis virescens larvae used are obtained from laboratory colonies. Pyrethroid-resistant H. virescens are derived from the PEG-strain.

Cotton leaves are immersed in solutions of the test compositions, or solutions of combinations of test compositions, for a period of about 3 seconds. Following immersion, leaves are allowed to air-dry for 2-3 hours. Plastic bioassay trays containing multiple open-faced faced wells (4.0×4.0×2.5 cm) are used as the test arenas. Cut portions of a treated leaf, a moistened cotton dental wick and a single third-instar larva are placed into each well, covered with an adhesive vented clear plastic sheet and held under constant fluorescent light at about 27° C. for a predetermined period of time. Larval mortality/morbidity is evaluated at 5 days after treatment. All treatments are replicated 4-5 fold in a randomized complete block design with 16-32 larvae per treatment. Using conventional log-probit analysis, the LC50 of each treatment is determined. These results allow comparison of test composition effectiveness between various insect species, as well as providing data that can be used to design compositions that can selectively affect the target insect species while minimally harming non-target species. The resulting data shows that the test compositions' efficacy is unaffected by insect resistance to pyrethroids, demonstrating that the mechanism of action employed by the test compositions differs from that employed by pyrethroids. Because of the difference in mechanisms of action, the test compositions are an effective component of a pest control regime designed to minimize resistance development in the target pest. Further, because the typical modes of resistance-development are less likely to result in resistance to the compositions of the invention, these compositions can delay, minimize, and prevent resistance development in the target pest.

### Example 11

Test compositions are mixed intimately with soil. The treated soil is filled into 250 ml pots and the pots are planted with pre-germinated broad beans. In this manner, the active compound can be taken up from the soil by the roots of the plants and transported into the leaves. To demonstrate the root-systemic effect, the leaves are populated with test insects after 7 days. After a further 7 days, the test is evaluated by determining percent mortality for the test insects. The data indicate that the test composition has a strongly pesticidal effect on the test insects, while having little or no effect on the broad beans. These results allow comparison of test composition effectiveness between various insect species, as well as providing data that can be used to design compositions that can selectively affect the target insect species while minimally harming non-target species.

### Example 12

After cultivating for three weeks, tomato plants are sprayed to drip point with an aqueous spray mixture prepared from test compositions, and 24 hours later they are infected with a suspension of a fungus (powdery mildew). Evaluation of the fungal attack takes place 5 days after infection, during which time conditions of 90 to 100 percent relative humidity and a temperature of 20° C. are maintained. Phytotoxicity data is also collected. The data shows that the test composition is effective against powdery mildew while causing no adverse effect on the tomato plants.

### Example 13

After cultivating for three weeks, an aqueous spray mixture prepared from a test composition is poured onto soybean plants. Care is taken that the spray mixture does not come into contact with the parts of the plants that are above ground. 48 hours later, the plants are infected with a suspension of a fungus (soybean rust). Evaluation of the fungal attack takes place 5 days after infection, during which time conditions of 90 to 100 percent relative humidity and a temperature of 20° C. are maintained. Phytotoxicity data is also collected. The data shows that the test composition is effective against soybean rust while causing no adverse effect on the soybean plants.

### Example 14

Six days after planting, wheat plants are sprayed to drip point with an aqueous spray mixture prepared from a test composition, and 24 hours later they are infected with a suspension of a fungus (flag smut). After an incubation period of 48 hours (conditions: 95 to 100 percent relative humidity at 20° C.), the plants are placed in a greenhouse at 22° C. 12 days after infection, the fungal attack is evaluated. Phytotoxicity data is also collected. The data shows that the test composition is effective against flag smut while causing no adverse effect on the wheat plants.

### Example 15

5 days after planting, an aqueous spray mixture prepared from a test composition is poured onto the soil surrounding wheat plants. Care is taken that the spray mixture does not come into contact with the parts of the plants that are above ground. 48 hours later, the plants are infected with a suspension of a fungus (stripe rust). After an incubation period of 48 hours (conditions: 95 to 100 percent relative humidity at 20%), the plants are placed in a greenhouse at 22° C. 12 days after infection, the fungal attack is evaluated. Phytotoxicity data is also collected. The data shows that the test composition is effective against stripe rust while causing no adverse effect on the wheat plants.

### Example 16

Young bean plants are colonized with a mixed population of Tetranychus urticae and are sprayed one day later with a aqueous emulsions spray mixture containing various concentrations of test compositions. The plants are subsequently incubated for 6 days at 25° C. and then evaluated. The percentage reduction of the population (% response) is determined by comparing the total number of dead eggs, larvae, and adults on the treated plants with those on the untreated plants. The data shows that the test composition is effective against Tetranychus urticae while causing no adverse effect on the bean plants.

### Example 17

Bush beans at the 2-leaf stage are colonised with a mixed population (eggs, larvae/nymphs, adults) of an insecticide-tolerant strain of Tetranychus cinnabarinus. 24 hours after infection, test compositions are applied to the plants from an automatic spray canister. The substances are ready-formulated and are diluted with water to the appropriate doses. The test is evaluated 2 and 7 days after application by the percentage mortality of eggs, larvae/nymphs and adults. The data indicate that the test composition has a strongly pesticidal effect on the insecticide-tolerant strain of Tetranychus cinnabarinus, while having little or no effect on the bush beans. These results allow comparison of test composition effectiveness between various insect species, as well as providing data that can be used to design compositions that can selectively affect the target insect species while minimally harming non-target species.

### Example 18

Numerous test compositions are tested for fungicidal activity in vivo against the diseases described below. The compounds are dissolved in a 1:1 mixture of acetone and methanol or N,N-dimethylformamide and diluted with a 2:1:1 mixture of water, acetone, and methanol (by volume) or water, respectively, to achieve the appropriate concentration. The solution is sprayed onto the plants, and allowed to dry (two hours). Then the plants are inoculated with fungal spores. Each test utilizes control plants which are sprayed with the appropriate solvent mixture and inoculated. For these protective tests, the plants are inoculated one day after treating the plants with the test composition. The remainder of the technique for each of the particular target species is described below.

### Wheat Leaf Rust (WLR)

Pucciitia recoitdita (f. sp. tritici) is cultured on 7-day old wheat (cultivar Fielder) over a 12-day period in a greenhouse. Spores are collected from the leaves by settling on aluminum foil. The spores are cleaned by sieving through a 250-micron opening screen and stored dry. The dried spores are used within one month. A spore suspension is prepared from dry uredia by adding 20 mg (9.5 million spores) per mL of Soltrol oil. The suspension is dispensed into gelatin capsules (0.7 mL capacity) which attach to the oil atomizers. One capsule is used per flat of twenty 2-inch square pots of 7-day old wheat plants, cultivar Fielder. After waiting for at least 15 minutes for the oil to evaporate from the wheat leaves, the plants are placed in a dark mist chamber (18-20° C. and 100% relative humidity) for 24 hours. The plants are then placed in the greenhouse and evaluated after 12 days for disease. The data shows that the test composition is effective against Pucciitia recoitdita while causing no adverse effect on the wheat plants.

### Wheat Leaf Blotch (SNW)

Cultures of Septoria nodorum are maintained on Czapek-Dox V-8 juice agar plates in an incubator at 20° C. with alternating periods of 12 hours of light and 12 hours of darkness for 2 weeks. A water suspension of the spores is obtained by shaking the portion of the plate with fungal material in deionized water and filtering through cheesecloth. The spore-containing water suspension is diluted to a spore concentration of 3×10⁶ spores per mL. The inoculum is dispersed by a DeVilbiss atomizer over one-week old Fielder wheat plants which have been previously sprayed with the test composition. The inoculated plants are placed in a humidity cabinet at 20° C. with alternating 12 hours of light and 12 hours of darkness for 7 days. The inoculated seedlings are then moved to a controlled environment room at 20° C. for 2 days of incubation. Disease control values are recorded as percent control. The data shows that the test composition is effective against Septoria nodorum while causing no adverse effect on the wheat plants.

### Wheat Powdery Mildew (WPM)

Erysiphe graminis (f. sp. tritici) is cultured on wheat seedlings, cultivar Fielder, in a controlled temperature room at 18° C. Mildew spores are shaken from the culture plants onto 7-day old wheat seedlings which have been previously sprayed with a test composition. The inoculated seedlings are kept in a controlled temperature room at 18° C. and sub-irrigated. The percent disease control is rated 7 days after the inoculation. The data shows that the test composition is effective against Erysiphe graminis while causing no adverse effect on the wheat plants.

### Cucumber Powdery Mildew (CPM)

Sphaerotheca fulginea is maintained on cucumber plants, cultivar. Bush Champion, in the greenhouse. Inoculum is prepared by placing five to ten 119 heavily mildewed cucumber leaves in a glass jar with 500 mL of water containing one drop of Tween 80 per 100 mL. After shaking the liquid and leaves, the inoculum is filtered through cheese cloth and misted onto the plants with a squirt bottle mister. The spore count is about 100,000 spores/mL. The plants are then placed in the greenhouse for infection and incubation. The plants are scored seven days after inoculation. Disease control values are recorded as percent control. The data shows that the test composition is effective against Sphaerotheca fulginea while causing no adverse effect on the cucumber plants.

### Example 19

Test compositions and conventional pesticides are compared to determine their respective ability to maintain pesticidal effectiveness over successive generations of pest species. For example, a wild-type strain of E. coli is treated with both penicillin (group 1) and a test composition from Table 1 or Table 2 (group 2). MIC (minimum inhibitory concentration; the lowest concentration of an antibacterial that will inhibit the visible growth of a microorganism after overnight incubation) values are determined at 1-week post-treatment for both groups, and the appropriate MIC amount is applied to both test populations. This is repeated for 4 weeks. The test data shows that the MIC value for group 1 increases over time, as the test population develops resistance to the compound. In contrast, the MIC value for group 2 remains static, indicating a lack of resistance development among the group 2 population.

1-25. (canceled)

26. A method of designing a selective pest control composition, comprising the steps of:
selecting a target pest with a first molecular target and a non-target organism with a second molecular target;
identifying at least two active agents for the composition, wherein the active agents in combination have a first activity upon the first molecular target in a target pest; and a second activity upon the second molecular target in the non-target organism, and
confirming that the at least two active agents have a synergistic effect upon the target pest and do not have a synergistic effect upon the non-target organism,
wherein the confirming step indicates that the composition is selective for the target pest over the non-target organism.

27. The method of claim 26, wherein the first molecular target is substantially absent in the non-target organism.

28. The method of claim 26, wherein the first molecular target is also present in the non-target organism.

29. The method of claim 26, wherein the first and second activities of the active agents are provided as LD₅₀.

30. The method of claim 29, wherein the ratio of LD₅₀ for the target pest relative to the LD₅₀ for the non-target organism is 0.5.

31. The method of claim 29, wherein the ratio of LD₅₀ for the target pest relative to the LD₅₀ for the non-target organism is 0.1.

32. The method of claim 29, wherein the ratio of LD₅₀ for the target pest relative to the LD₅₀ for the non-target organism is 0.05.

33. The method of claim 29, wherein the ratio of LD₅₀ for the target pest relative to the LD₅₀ for the non-target organism is 0.02.

34. The method of claim 29, wherein the ratio of LD₅₀ for the target pest relative to the LD₅₀ for the non-target organism is 0.01.

35. The method of claim 26, wherein the confirming step comprises confirming that the agents in combination have an effect in the non-target organism selected from the group consisting of: an antagonistic effect, a non-additive effect, and an additive effect.

36. The method of claim 26, wherein the first molecular target is a G-protein coupled receptor.

37. The method of claim 36, wherein the G-protein coupled receptor is selected from the group consisting of: a tyramine receptor, an octopamine receptor, olfactory receptor Or83b and olfactory receptor 43a.

38. A method of developing a selective pest control composition, comprising the steps of:
selecting a target pest for selective control by the composition, and a non-target organism to be substantially unaffected by the composition;
identifying at least two active agents for the composition, wherein the active agents in combination have a synergistic effect within the target pest; and
confirming that the identified active agents do not have a synergistic effect within the non-target organism, to establish that the composition is selective of the target pest over the non-target organism.

39. A method of designing a selective pest control composition, comprising the steps of:
selecting a target pest for selective control by the composition and a non-target organism to be substantially unaffected by the composition;
identifying at least two active agents for the composition, wherein the active agents in combination have a first activity upon the target pest; and a second activity upon the non-target organism, and
confirming that the at least two active agents have a synergistic effect upon the target pest and do not have a synergistic effect upon the non-target organism,
wherein the confirming step indicates that the composition is selective for the target pest over the non-target organism.

40. A method of developing a low-resistance pest control foiuiulation against a target pest, comprising the steps of
selecting a target pest, the pest having at least a first and a second molecular target, where the molecular targets are under genetic control;
selecting at least two active agents, wherein a first active agent interacts with the first molecular target under genetic control and a second active agent interacts with the second molecular target under genetic control; and
combining the two active agents in a formulation, wherein the agents in the formulation act upon the target pest in a synergistic manner, and wherein resistance to the formulation in an individual target pest requires two separate genetic lesions divergent from a non-resistant population of the pest.
